(19) United States
(12) Patent Application Publication
(10) Pub. No.: US 2012/0071669 A1
(43) Pub. Date: Mar. 22, 2012

(54) PROCESS FOR PREPARATION OF ELETRIPTAN AND SALT THEREOF

(75) Inventors: Dhananjay Govind Sathe, Maharashtra (IN); Subhash Vishwanath Damle, Maharashtra (IN); Subodh Shashikant Patnekar, Maharashtra (IN); Prashant Vijay Joshi, Maharashtra (IN); Samadhan Daulat Patil, Maharashtra (IN)

(73) Assignee: USV Limited B.S.D. Mar, Mumbai (IN)

(21) Appl. No.: 13/183,911

(22) Filed: Jul. 15, 2011

(30) Foreign Application Priority Data
Jul. 15, 2010 (IN) 2035/MUM/2010

(51) Int. Cl. C07D 403/06

(52) U.S. Cl. 548/468

(57) ABSTRACT

The present invention relates to an improved process for the preparation of 3-(N-methyl-2(R)-pyrrolidinyl methyl)-5-[2-(phenyl sulfonyl)ethyl]-1H-indole or pharmaceutically acceptable salts thereof, particularly 3-(N-methyl-2(R)-pyrrolidinyl methyl)-5-[2-(phenyl sulfonyl)ethyl]-1H-indole hydrobromide (Eletriptan hydrobromide). The present invention further relates to novel polymorphs of 3-(N-methyl-2(R)-pyrrolidinyl methyl)-5-[2-(phenyl sulfonyl)ethenyl]-1H-indole hydrobromide and process for preparation thereof.

### RELATED APPLICATION

This application claims priority from Indian Application Serial No. 2035/MUM/2010 filed Jul. 15, 2010, the contents of which are incorporated herein by reference in its entirety.

### FIELD OF INVENTION

The present invention relates to an improved process for the preparation of 3-(N-methyl-2(R)-pyrrolidinyl methyl)-5-[2-(phenyl sulfonyl)ethyl]-1H-indole or pharmaceutically acceptable salts thereof, particularly 3-(N-methyl-2(R)-pyrrolidinyl methyl)-5-[2-(phenyl sulfonyl)ethyl]-1H-indole hydrobromide (Eletriptan hydrobromide). The present invention further relates to novel polymorphs of 3-(N-methyl-2(R)-pyrrolidinyl methyl)-5-[2-(phenyl sulfonyl)ethenyl]-1H-indole hydrobromide and process for preparation thereof.

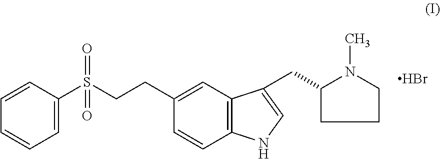

### BACKGROUND OF INVENTION

Eletriptan hydrobromide is chemically designated as 3-[[(2R)-1-Methyl-2-pyrrolidinyl]methyl]-5-[2-(phenylsulfonyl)ethyl]-1H-indole monohydrobromide or 3-(N-methyl-2(R)-pyrrolidinyl methyl)-5-[2-(phenyl sulfonyl)ethyl]-1H-indole hydrobromide. Eletriptan is marketed by Pfizer under the brand name RELPAX®. Each RELPAX° Tablet for oral administration contains 24.2 or 48.5 mg of Eletriptan hydrobromide equivalent to 20 mg or 40 mg of eletriptan respectively. It is administered as single doses of 20 mg and 40 mg. RELPAX® is a prescription medicine used to treat migraine headaches with or without aura in adults. Migraine is an intense, throbbing headache and the visual symptoms before the headache such as flashing lights or wavy lines are called an aura. RELPAX® reduces swelling of blood vessels surrounding the brain. This swelling is associated with the headache pain of migraine attack. Eletriptan blocks the release of substances from nerve endings that cause more pain and other symptoms like nausea and sensitivity to light and sound. It is thought that these actions contribute to relief of symptoms by Eletriptan.

Eletriptan is a selective 5-hydroxytryptamine 1B/1D (5-HT 1B/1D) receptor agonist. It binds with high affinity to 5-HT[1B, 1D, 1F] receptors. It has modest affinity to 5-HT[1A, 1E, 2B, 7] receptors and little or no affinity for 5-HT[2A, 2C, 3, 4, 5A, 6] receptors. It has no significant affinity or pharmacological activity at adrenergic alpha₁, alpha₂, or beta; dopaminergic D₁ or D₂; muscarinic; or opioid receptors. Two theories have been proposed to explain the efficacy of 5-HT receptor agonists in migraine. One theory suggests that activation of 5-HT, receptors located on intracranial blood vessels, including those on the arteriovenous anastomoses, leads to vasoconstriction, which is correlated with the relief of migraine headache. The other hypothesis suggests that activation of 5-HT, receptors on sensory nerve endings in the trigeminal system results in the inhibition of pro-inflammatory neuropeptide release.

U.S. Pat. No. 5,545,644 discloses indole derivatives and process for preparation thereof. The process for preparation of Eletriptan as described in US '644 comprises, treating N-benzyloxycarbonyl-D-proline with oxalyl chloride in methylene dichloride to get N-benzyloxycarbonyl-D-proline acid chloride. A solution of ethyl magnesium bromide is added to a solution of 5-bromoindole in diethyl ether to form indole magnesium bromide. The solution of N-benzyloxycarbonyl-D-proline acid chloride in methylene dichloride is added to the obtained solution to get (R)-3-(N-benzyloxycarbonyl-pyrrolidin-2-ylcarbonyl)-5-bromo-1H-indole which is chromatographed on silica gel. The obtained compound is then reduced using lithium aluminium hydride in tetrahydrofuran to get (R)-5-bromo-3-(N-methylpyrrolidin-2-ylmethyl)-1H-indole, which is chromatographed on silica gel. The purified N-methyl compound is coupled with phenyl vinyl sulfone in presence of triethylamine using palladium (II) acetate and tri-o-tolyl phosphine in acetonitrile under reflux for 42 hours in an atmosphere of nitrogen to get the residue of (R)-5-(2-Benzenesulphonylethenyl)-3-(N-methylpyrrolidin-2-ylmethyl)-1H-indole hydrobromide which is chromatographed on silica gel to get a foamy compound. The obtained compound is reduced using 10% palladium on carbon in a mixture of absolute ethanol, dimethylformamide and water under hydrogen atmosphere at room temperature for 18 hours. The obtained residue is treated with 2M aqueous sodium carbonate solution to get a gummy residue of the Eletriptan which is chromatographed on silica gel. The reaction scheme is represented in Scheme 1,

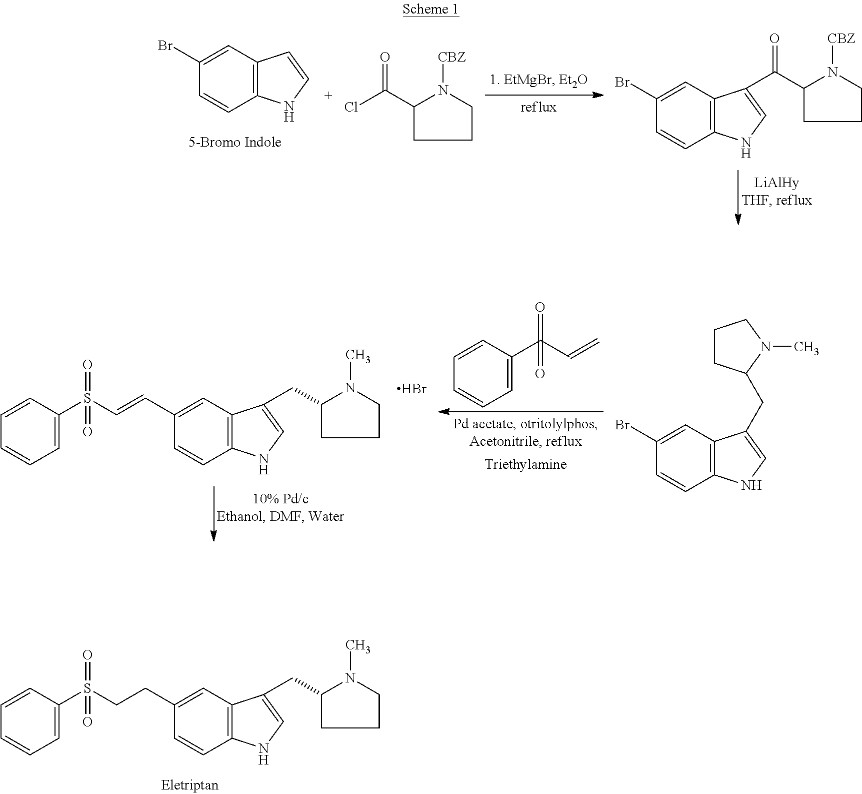

U.S. Pat. No. 6,110,940 discloses polymorphic alpha form and beta form of Eletriptan hydrobromide and process for preparation thereof. US '940 discloses various methods for preparation of alpha form of Eletriptan hydrobromide, viz.,

a) treatment of a solution of 3-(N-methyl-2(R)-pyrrolidinylmethyl)-5-(2-phenylsulphonylethyl)-1H-indole in a suitable solvent, preferably acetone, at room temperature, with an aqueous solution of hydrogen bromide, followed by crystallisation of the isolated crude oil from a suitable solvent, preferably 2-propanol, affords the alpha form of the required hydrobromide salt, or
b) treatment of a solution of 3-(N-methyl-2(R)-pyrrolidinylmethyl)-5-(2-phenylsulphonylethyl)-1H-indole in a suitable solvent, preferably acetone or an ether solvent such as tetrahydrofuran or 1,2-dimethoxyethane, more preferably 1,2-dimethoxyethane, at from 0 to 10° C. with an aqueous solution of hydrogen bromide, furnishes the beta-form of the required hydrobromide salt, or
c) crystallisation of the beta form of Eletriptan hydrobromide from a suitable solvent, preferably aqueous acetone, followed by slurrying of the resulting mixture, gives the desired alpha form, or
d) treatment of a solution of 3-(N-methyl-2(R)-pyrrolidinylmethyl)-5-(2-phenylsulphonylethyl)-1H-indole in a suitable solvent, preferably acetone, at from 0 to 5° C. with an aqueous solution of hydrogen bromide and then slurrying of the reaction mixture, optionally followed by heating under reflux, cooling and further slurrying, provides the required alpha form.

WO2008137134 describes amorphous form of eletriptan hydrobromide (ELT-HBr) and process for the preparation thereof. WO '134 also discloses various processes for preparation of alpha and beta form of Eletriptan hydrobromide viz.,

a) slurrying ELT-HBr Form [beta] in a solvent selected from a group consisting of isobutanol, methylacetate, mixtures of THF and water, and cyclohexane, wherein the mixture of water and THF contains from about 0.5% of water by volume to about 2% of water by volume, or
b) crystallizing ELT-HBr from ethanol from a solution of ELT-HBr in ethanol, and precipitating crystalline ELT-HBr alpha Form, or
c) heating wet crystalline ELT-HBr Form beta wherein the heating is conducted at a temperature of about 45° C. to about 60° C. under reduced pressure of about 10 to about 30 mm Hg.

WO2009142771 describes a method of purifying Eletriptan and its intermediates comprising preparing the BIP (5-bromo-3-[(R)-1-methyl-pyrrolidin-2-ylmethyl]-1H-indole) salts and converting them to Eletriptan and salts thereof. WO '771 also discloses process for preparation of Eletriptan hydrobromide by converting Eletriptan PTSA salt to Eletriptan base followed by treatment of obtained base with HBr to get Eletriptan hydrobromide.

WO2010049952 discloses the method for preparing Eletriptan, comprising the steps of coupling 5-Bromo indole with phenyl vinyl sulfone under Heck reaction conditions followed by acylation with Cbz-Proline acid chloride to obtain N-protected Cbz intermediate, which on reduction in presence of a hydride agent provides Eletriptan.

WO2010121673 describes a synthetic process for Eletriptan or its salt, comprising salifying 5-bromo indole derivative using dicarboxylic acid to obtain the corresponding salt followed by optionally purifying the obtained salt by solvent crystallization to obtain the purified salt and converting said salt to 1-(3-{[(2R)-1-methylpyrrolidin-2yl]methyl}-5-[(E)-2-(phenylsulfonyl)ethenyl]-1H-indole-1-yl)ethanone, which is converted into Eletriptan or its salt.

WO2011004391 discloses an improved process for Eletriptan hydrobromide which involves the hydrolysis of (R)-1-acetyl-5-(2-phenylsulphonylethenyl)-3-(N-methylpyrrolidine-2-ylmethyl)-1H-indole followed by reduction in the presence of metal catalyst and methanesulphonic acid to get Eletriptan methanesulphonate, which is further converted to Eletriptan hydrobromide.

### Disadvantages of the Prior Art Processes:

1. Requires longer reaction time and column chromatography in all steps.
2. The process is time consuming and not economical.
3. Low yield and low purity.

In view of the disadvantages associated with the prior art processes for preparation of Eletriptan and pharmaceutically acceptable salts thereof, there is a need for an improved process for the preparation of Eletriptan and its salt, particularly hydrobromide salt in high yield and purity. The present invention provides simple, cost effective, consistent and industrially feasible process for preparation of Eletriptan hydrobromide.

### OBJECT OF THE INVENTION

An object of the present invention is to provide a simple, cost effective, consistent and industrially feasible process for the preparation of Eletriptan or pharmaceutically acceptable salts thereof, in particular Eletriptan hydrobromide.

Another object of the present invention is to provide novel polymorphs of 3-(N-methyl-2(R)-pyrrolidinyl methyl)-5-[2-(phenyl sulfonyl)ethenyl]-1H-indole hydrobromide and process for preparation thereof.

Another object of the present invention provides Eletriptan hydrobromide substantially free from impurity selected from the group consisting of 2-Chloroethylphenyl sulphide (A), 2-Chloroethylphenyl sulphone (B), phenyl vinyl sulfone (C), N-benzyloxycarbonyl-D-proline acid chloride (D), 3-(N-methyl-2(R)-pyrrolidinyl methyl)-5-[2-(phenylsulfonyl)ethenyl]-1H-indole (E), 3-(1-Methyl pyrrolidin-2(R)-yl-methyl)-1H-indole (F) and 3-[N-Methyl-2(R)-pyrrolidinylmethyl]-1,5-bis-(2-phenylsulfonylethyl)indole (G).

Yet another object of the present invention provides process for the preparation of 3-[N-Methyl-2(R)-pyrrolidinylmethyl]-1,5-bis-(2-phenylsulfonylethyl)indole.

Another object of the invention is to provide pharmaceutical compositions comprising Eletriptan or pharmaceutically acceptable salts thereof and at least one pharmaceutically acceptable excipient.

### SUMMARY OF THE INVENTION

According to one aspect of the present invention, there is provided a process for preparation of Eletripan hydrobromide comprising,
a) coupling 3-(N-methyl-2(R)-pyrrolidinylmethyl)-5-bromo-1H-indole with phenyl vinyl sulfone to obtain a mixture containing 3-(N-methyl-2(R)-pyrrolidinyl methyl)-5-[2-(phenyl sulfonyl)ethenyl]-1H-indole hydrobromide;
b) isolating 3-(N-methyl-2(R)-pyrrolidinyl methyl)-5-[2-(phenylsulfonyl)ethenyl]-1H-indole hydrobromide as a solid;
c) converting said solid 3-(N-methyl-2(R)-pyrrolidinyl methyl)-5-[2-(phenylsulfonyl)ethenyl]-1H-indole hydrobromide to 3-(N-methyl-2(R)-pyrrolidinylmethyl)-5-[2-(phenylsulfonyl)ethyl]-1H-indole hydrobromide (Eletriptan hydrobromide).

Preferably, coupling in step a) is carried out at a temperature of 70 to 85° C. for 6 to 14 hours in presence of an organopalladium catalyst selected from palladium acetate, palladium chloride or tetrakis(phenylphosphine)palladium(0); a triarylphosphine selected from tri-o-tolylphosphine, triphenylphosphine or BINAP; base selected from triethylamine, trimethyl amine, potassium carbonate, sodium carbonate, sodium acetate or potassium acetate; and solvent selected from acetonitrile, N,N-dimethylformamide or tetrahydrofuran.

Another aspect of the present invention provides the isolation process of 3-(N-methyl-2(R)-pyrrolidinylmethyl)-5-[2-(phenylsulfonyl)ethenyl]-1H-indole hydrobromide from the reaction mixture comprising the steps of,

a) filtering said mixture containing 3-(N-methyl-2(R)-pyrrolidinylmethyl)-5-[2-(phenylsulfonyl)ethenyl]-1H-indole hydrobromide followed by concentrating the filtrate under vacuum to obtain a residue;
b) treating said residue with a solvent to obtain 3-(N-methyl-2(R)-pyrrolidinylmethyl)-5-[2-(phenylsulfonyl)ethenyl]-1H-indole hydrobromide as solid;
c) optionally, purifying said obtained 3-(N-methyl-2(R)-pyrrolidinylmethyl)-5-[2-(phenylsulfonyl)ethenyl]-1H-indole hydrobromide.

Preferably, solvent used in step b) is a polar aprotic solvent is selected from acetonitrile, N,N-dimethylformamide, dimethylsulfoxide, dichloromethane, ethyl acetate, acetone or tetrahydrofuran.

Preferably, purification of 3-(N-methyl-2-(R)-pyrrolidinylmethyl)-5-[2-(phenyl sulfonyl)ethenyl]-1H-indole hydrobromide is carried out by a process comprising the steps of,

a) dissolving 3-(N-methyl-2-(R)-pyrrolidinylmethyl)-5-[2-(phenyl sulfonyl)ethenyl]-1H-indole hydrobromide in first solvent to obtain a solution;
b) optionally adding second solvent to said solution to obtain a mixture; and
c) isolating pure 3-(N-methyl-2-(R)-pyrrolidinylmethyl)-5-[2-(phenyl sulfonyl)ethenyl]-1H-indole hydrobromide from said solution or said mixture.

Preferably, first solvent is selected from toluene, xylene, benzene, hexane, heptane, halogenated derivative thereof, methanol, ethanol, isopropanol, n-propanol, butanol or a mixture thereof; and said second solvent is selected from methanol, ethanol, isopropanol, n-propanol, butanol, dichloromethane, N,N-dimethylformamide, ethyl acetate, acetonitrile, tetrahydrofuran, dimethylsulfoxide or mixture thereof.

Another aspect of the present invention provides 3-(N-methyl-2-(R)-pyrrolidinylmethyl)-5-[2-(phenyl sulfonyl)ethenyl]-1H-indole hydrobromide characterized by X-ray diffraction pattern having peaks at 2-theta values of about 11.47, 12.63, 14.41, 15.09, 18.42, 19.77, 22.22, 23.18, 23.90, 25.23, 26.94, 28.33, 29.90 and 30.85 degrees; or by X-ray diffraction pattern having peaks at 2-theta values of about 10.12, 11.34, 13.87, 15.63, 16.09, 16.43, 18.32, 19.95, 20.39, 20.90, 21.82, 22.75, 24.16, 24.37, 24.93, 25.69, 27.47, 27.91 and 28.85 degrees.

Another aspect of the present invention provides the conversion of 3-(N-methyl-2-(R)-pyrrolidinylmethyl)-5-[2-(phenylsulfonyl)ethenyl]-1H-indole hydrobromide to 3-(N-methyl-2(R)-pyrrolidinylmethyl)-5-[2-(phenylsulfonyl)ethyl]-1H-indole hydrobromide (Eletriptan hydrobromide) comprising the steps of,

a) subjecting 3-(N-methyl-2-(R)-pyrrolidinylmethyl)-5-[2-(phenylsulfonyl)ethenyl]-1H-indole hydrobromide to reduction to obtain 3-(N-methyl-2(R)-pyrrolidinylmethyl)-5-[2-(phenylsulfonyl)ethyl]-1H-indole hydrobromide;
b) treating 3-(N-methyl-2(R)-pyrrolidinylmethyl)-5-[2-(phenylsulfonyl)ethyl]-1H-indole hydrobromide obtained in step (a) with HBr in a solvent selected from isopropanol, n-propanol, ethanol, methanol, n-butanol or mixture thereof to obtain 3-(N-methyl-2(R)-pyrrolidinylmethyl)-5-[2-(phenyl sulfonyl)ethyl]-1H-indole hydrobromide with the required HBr content;
c) optionally purifying 3-(N-methyl-2(R)-pyrrolidinylmethyl)-5-[2-(phenyl sulfonyl)ethyl]-1H-indole hydrobromide to obtain pure 3-(N-methyl-2(R)-pyrrolidinylmethyl)-5-[2-(phenylsulfonyl)ethyl]-1H-indole hydrobromide.

Preferably, reduction is carried out at a temperature of about 30° C. to 50° C. and pressure of 5 Kg/cm² for 6 to 10 hours in the presence of hydrogen gas; catalyst selected from palladium, platinum oxide, rhodium, ruthenium or Raney nickel; and solvent selected from methanol, ethanol, n-propanol or isopropanol.

Another aspect of the present invention provides a process for preparation of Eletriptan hydrobromide, wherein the process comprises, treating 3-(N-methyl-2(R)-pyrrolidinylmethyl)-5-[2-(phenylsulfonyl)ethyl]-1H-indole hydrobromide having inadequate HBr content with HBr to obtain Eletriptan hydrobromide with required hydrobromide content. Preferably HBr is used as HBr/isopropanol (IPA) in the concentration of 10 to 30% HBr, preferably 15 to 25% HBr in IPA.

Another aspect of the present invention provides a process for purification of Eletriptan hydrobromide comprising the steps of

a) dissolving 3-(N-methyl-2(R)-pyrrolidinyl methyl)-5-[2-(phenyl sulfonyl)ethyl]-1H-indole hydrobromide (Eletriptan hydrobromide) in solvent or mixture of solvents to get a clear solution;
b) optionally treating the obtained clear solution with neutral alumina and/or activated charcoal to get a mixture; and
c) isolating pure 3-(N-methyl-2(R)-pyrrolidinyl methyl)-5-[2-(phenyl sulfonyl)ethyl]-1H-indole hydrobromide (Eletriptan hydrobromide) substantially free of impurities.

Preferably, solvent is selected from acetone, methylethylketone, methyl isobutyl ketone, methanol, ethanol, n-propanol, isopropanol, butanol or mixture thereof.

Another aspect of the present invention provides 3-[N-Methyl-2(R)-pyrrolidinyl methyl]-1,5-bis-(2-phenylsulfonylethyl)indole prepared by a process comprising treating 3-(N-methyl-2(R)-pyrrolidinylmethyl)-5-[2-(phenylsulfonyl)ethyl]-1H-indole (Eletriptan base) with phenylvinyl sulfone in presence of a base.

Another aspect of the present invention provides use of 3-[N-Methyl-2(R)-pyrrolidinyl methyl]-1,5-bis-(2-phenylsulfonylethyl)indole as a reference marker and/or reference standard in testing the purity of a sample of Eletriptan or a pharmaceutical dosage form comprising Eletriptan.

### BRIEF DESCRIPTION OF THE DRAWINGS

FIG. 1: X-ray diffraction pattern of 3-(N-methyl-2(R)-pyrrolidinylmethyl)-5-[2-(phenylsulfonyl)ethenyl]-1H-indole hydrobromide.

FIG. 2: IR spectrum of 3-(N-methyl-2(R)-pyrrolidinyl methyl)-5-[2-(phenyl sulfonyl)ethenyl]-1H-indole hydrobromide.

FIG. 3: X-ray diffraction pattern of 3-(N-methyl-2(R)-pyrrolidinylmethyl)-5-[2-(phenylsulfonyl)ethenyl]-1H-indole hydrobromide.

FIG. 4: X-ray diffraction pattern of 3-(N-methyl-2(R)-pyrrolidinylmethyl)-5-[2-(phenyl sulfonyl)ethenyl]-1H-indole hydrobromide (amorphous).

FIG. 5: IR spectrum of X-ray diffraction pattern of 3-(N-methyl-2(R)-pyrrolidinyl methyl)-5-[2-(phenylsulfonyl)ethenyl]-1H-indole hydrobromide.

FIG. 6: DSC of X-ray diffraction pattern of 3-(N-methyl-2(R)-pyrrolidinyl methyl)-5-[2-(phenylsulfonyl)ethenyl]-1H-indole hydrobromide.

FIG. 7: X-ray diffraction pattern of Eletriptan hydrobromide obtained according to the present invention.

FIG. 8: DSC of Eletriptan hydrobromide obtained according to the present invention.

FIG. 9: IR spectrum of Eletriptan hydrobromide obtained according to the present invention.

### DETAILED DESCRIPTION OF THE INVENTION

The present invention provides an improved process for the preparation of 3-(N-methyl-2(R)-pyrrolidinyl methyl)-5-[2-(phenyl sulfonyl)ethyl]-1H-indole (Eletriptan) or pharmaceutically acceptable salts thereof, particularly hydrobromide.

According to one embodiment of the present invention, the process for preparation of Eletriptan hydrobromide comprises the steps of,

a) coupling 3-(N-methyl-2(R)-pyrrolidinylmethyl)-5-bromo-1H-indole (III) with phenyl vinyl sulfone (IV) in suitable solvent to obtain a mixture of 3-(N-methyl-2(R)-pyrrolidinylmethyl)-5-[2-(phenylsulfonyl)ethenyl]-1H-indole hydrobromide (II);
b) isolating 3-(N-methyl-2(R)-pyrrolidinylmethyl)-5-[2-(phenyl sulfonyl)ethenyl]-1H-indole hydrobromide as a solid;
c) converting said solid 3-(N-methyl-2(R)-pyrrolidinylmethyl)-5-[2-(phenyl sulfonyl)ethenyl]-1H-indole hydrobromide to 3-(N-methyl-2(R)-pyrrolidinylmethyl)-5-[2-(phenyl sulfonyl)ethyl]-1H-indole hydrobromide.

In a preferred embodiment, the process for preparation of Eletriptan hydrobromide comprises the preparation of 3-(N-methyl-2(R)-pyrrolidinylmethyl)-5-[2-(phenyl sulfonyl)ethenyl]-1H-indole hydrobromide by Mizoroki Heck reaction, which comprises coupling 3-(N-methyl-2(R)-pyrrolidinylmethyl)-5-bromo-1H-indole (III) with phenyl vinyl sulfone (IV) in the presence of an organopalladium catalyst, a triarylphosphine and a base in a solvent to get a reaction mixture. The obtained reaction mixture is refluxed for 6-14 hours, preferably for 8-12 hours at temperature of about 70-85° C., preferably about 80-82° C. to get a mixture containing 3-(N-methyl-2(R)-pyrrolidinyl methyl)-5-[2-(phenyl sulfonyl)ethenyl]-1H-indole hydrobromide (II); filtering the mixture containing 3-(N-methyl-2(R)-pyrrolidinylmethyl)-5-[2-(phenyl sulfonyl)ethenyl]-1H-indole hydrobromide (II) followed by concentrating the filtrate under vacuum to obtain a residue; treating the obtained residue with a solvent to obtain 3-(N-methyl-2(R)-pyrrolidinylmethyl)-5-[2-(phenyl sulfonyl)ethenyl]-1H-indole hydrobromide (II) as a solid; optionally purifying the obtained 3-(N-methyl-2(R)-pyrrolidinylmethyl)-5-[2-(phenyl sulfonyl) ethenyl]-1H-indole hydrobromide (II) using a solvent to obtain pure 3-(N-methyl-2(R)-pyrrolidinylmethyl)-5-[2-(phenyl sulfonyl)ethenyl]-1H-indole hydrobromide(II); and converting 3-(N-methyl-2(R)-pyrrolidinylmethyl)-5-[2-(phenyl sulfonyl)ethenyl]-1H-indole hydrobromide (II) to 3-(N-methyl-2(R)-pyrrolidinyl methyl)-5-[2-(phenyl sulfonyl)ethyl]-1H-indole hydrobromide (Eletriptan hydrobromide).

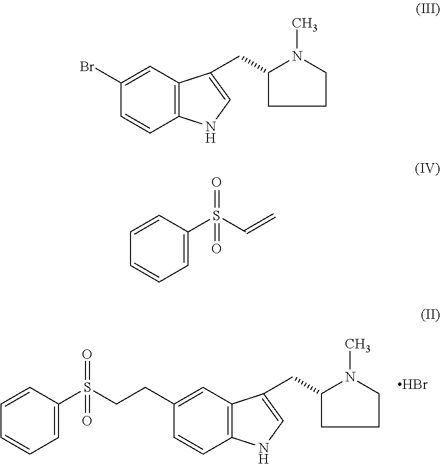

Organopalladium catalyst include tetrakis(triphenylphosphine)palladium(0) and palladium(II) salts selected from preferably palladium(II) acetate or palladium chloride. Suitable solvent is selected from acetonitrile, N,N-dimethylformamide, tetrahydrofuran, 1,2-dimethoxyethane or mixture thereof, preferred solvent being acetonitrile or N,N-dimethylformamide. Triarylphosphine is selected from tri-o-tolylphosphine, triphenylphosphine or BINAP. Base is selected from triethylamine, trimethyl amine, potassium carbonate, sodium carbonate, potassium acetate, sodium acetate, preferably triethylamine.

Prior art discloses that reaction mixture is refluxed for 42 hours. It has been surprisingly found by the inventors of the present invention that the completion of the reaction can be achieved in about 8-12 hours.

The solvent treatment of 3-(N-methyl-2(R)-pyrrolidinylmethyl)-5-[2-(phenyl sulfonyl)ethenyl]-1H-indole hydrobromide(II) is carried out for removal of traces of triethylamine which involves stripping of 3-(N-methyl-2(R)-pyrrolidinylmethyl)-5-[2-(phenyl sulfonyl)ethenyl]-1H-indole hydrobromide(II) with a suitable solvent and/or adding a suitable solvent to 3-(N-methyl-2(R)-pyrrolidinylmethyl)-5-[2-(phenyl sulfonyl)ethenyl]-1H-indole hydrobromide(II) followed by isolation of 3-3-(N-methyl-2(R)-pyrrolidinylmethyl)-5-[2-(phenyl sulfonyl)ethenyl]-1H-indole hydrobromide (II) from the mixture by filtration.

Suitable solvent is selected from acetonitrile, N,N-dimethylformamide, dimethylsulfoxide, dichloromethane, ethyl acetate, acetone, or tetrahydrofuran, preferably acetontirile.

Prior art reports that 3-(N-methyl-2(R)-pyrrolidinylmethyl)-5-[2-(phenyl sulfonyl)ethenyl]-1H-indole hydrobromide (II) is obtained as foamy compound. The present invention provides 3-(N-methyl-2(R)-pyrrolidinylmethyl)-5-[2-(phenyl sulfonyl)ethenyl]-1H-indole hydrobromide (II) as a solid which is easy to handle and purify. The process of the present invention reduces the possible impurities in the initial stage thereby providing the final product (Eletriptan) in high yield and purity.

According to another embodiment of the present invention, 3-(N-methyl-2(R)-pyrrolidinylmethyl)-5-[2-(phenyl sulfonyl)ethenyl]-1H-indole hydrobromide (II) is purified by a process comprising the steps of,

a) dissolving 3-(N-methyl-2(R)-pyrrolidinylmethyl)-5-[2-(phenyl sulfonyl)ethenyl]-1H-indole hydrobromide (II) in a first solvent to obtain a first mixture;
b) optionally, adding second solvent to the obtained first mixture to obtain a second mixture; and
c) isolating pure 3-(N-methyl-2(R)-pyrrolidinylmethyl)-5-[2-(phenyl sulfonyl)ethenyl]-1H-indole hydrobromide (II) from first mixture or second mixture.

In a preferred embodiment, 3-(N-methyl-2(R)-pyrrolidinylmethyl)-5-[2-(phenyl sulfonyl)ethenyl]-1H-indole hydrobromide (II) is purified using a solvent or a mixture of solvents. 3-(N-methyl-2(R)-pyrrolidinylmethyl)-5-[2-(phenyl sulfonyl)ethenyl]-1H-indole hydrobromide(II) is dissolved in a first solvent selected from hydrocarbon, alcohol or mixture thereof at temperature in the range of 30° C. to reflux temperature of the solvent, preferably 65-75° C., more preferably 70° C. to get a solution, followed by addition of a second solvent selected from alcohol, polar aprotic solvent or mixture thereof to obtain a mixture. The obtained mixture is stirred at the same temperature for 1-3 hrs, preferably for 2 hours. The mixture is then cooled and filtered to get a solid.

Hydrocarbon is selected from toluene, xylene, benzene, hexane, heptane, halogenated derivative thereof or mixture thereof. Alcohol is selected from methanol, ethanol, isopropanol (IPA), n-propanol, butanol or mixture thereof. Polar aprotic solvent is selected from acetonitrile, dichloromethane, N,N-dimethylformamide, ethyl acetate, acetone, tetrahydrofuran or dimethylsulfoxide. Preferably the first solvent is toluene and the second solvent is methanol or ethanol.

Prior art discloses the use of column chromatography for the purification of 3-(N-methyl-2(R)-pyrrolidinylmethyl)-5-[2-(phenyl sulfonyl)ethenyl]-1H-indole hydrobromide (II). The present invention avoids the use of column chromatography. The commercial use of column chromatography is not viable at industrial scale as it is time consuming and tedious. 3-(N-methyl-2(R)-pyrrolidinylmethyl)-5-[2-(phenyl sulfonyl)ethenyl]-1H-indole hydrobromide (II) obtained after column chromatography has purity of 70% to 80% rendering loss of 25% to 30% of the yield in the final product due to presence of closely eluting impurities, which is not feasible. The present invention uses organic solvents for the purification of 3-(N-methyl-2(R)-pyrrolidinylmethyl)-5-[2-(phenyl sulfonyl)ethenyl]-1H-indole hydrobromide (II), which makes the process not only commercially viable but also gives substantially pure intermediate 3-(N-methyl-2(R)-pyrrolidinylmethyl)-5-[2-(phenyl sulfonyl)ethenyl]-1H-indole hydrobromide (II) which makes the process cost effective. Many of the impurities are removed at the purification step yielding 3-(N-methyl-2(R)-pyrrolidinylmethyl)-5-[2-(phenyl sulfonyl)ethenyl]-1H-indole hydrobromide (II) with purity of above 90% thus making the process cost effective and reducing the loss of yield in the final stage.

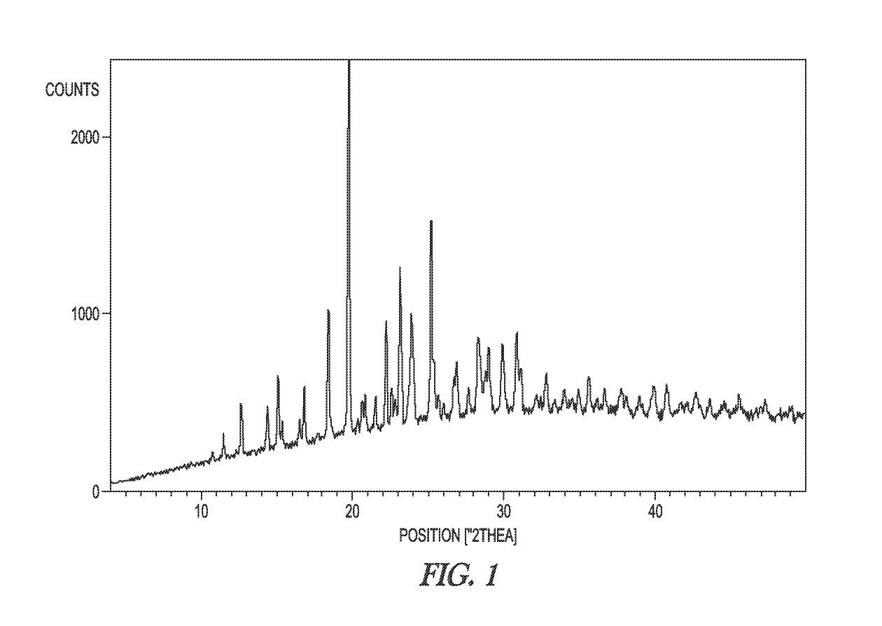

In a preferred embodiment of the present invention, 3-(N-methyl-2(R)-pyrrolidinylmethyl)-5-[2-(phenyl sulfonyl)ethenyl]-1H-indole hydrobromide (II) is obtained in a crystalline form characterized by X-ray diffraction pattern as shown in FIG. 1. The crystalline form of 3-(N-methyl-2(R)-pyrrolidinylmethyl)-5-[2-(phenyl sulfonyl)ethenyl]-1H-indole hydrobromide (II) is characterized by X-ray powder diffraction (XRPD) having characteristic peaks expressed as 2-theta values of about 11.47, 12.63, 14.41, 15.09, 18.42, 19.77, 22.22, 23.18, 23.90, 25.23, 26.94, 28.33, 29.90 and 30.85 degrees. It is further characterized by peaks expressed as 2-theta of about 16.82, 20.65, 20.89, 21.56, 22.60, 27.73, 29.02, 32.81, 35.64, 36.70, 39.02, 39.92, 40.85, 42.72, 43.64 and 47.35 degrees.

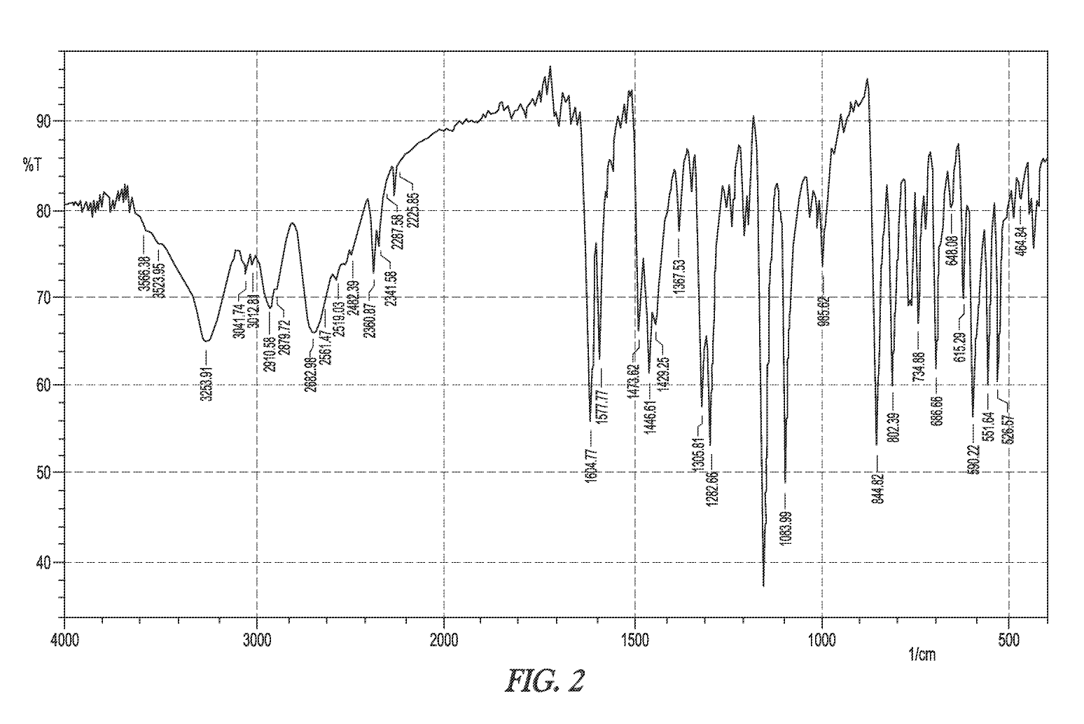

Yet another embodiment of the present invention provides crystalline form of 3-(N-methyl-2(R)-pyrrolidinylmethyl)-5-[2-(phenyl sulfonyl)ethenyl]-1H-indole hydrobromide (II), characterized by an infra-red (IR) spectrum as shown in FIG. 2. The crystalline form of 3-(N-methyl-2(R)-pyrrolidinylmethyl)-5-[2-(phenyl sulfonyl)ethenyl]-1H-indole hydrobromide(II) is characterized by an infra-red spectrum showing bands at 3254, 3042, 2910, 2683, 2361, 2341, 1605, 1578, 1474, 1447, 1429, 1306, 1283, 1144, 1084, 986, 845, 802, 735, 687, 615, 590, 552 and 527 cm⁻¹.

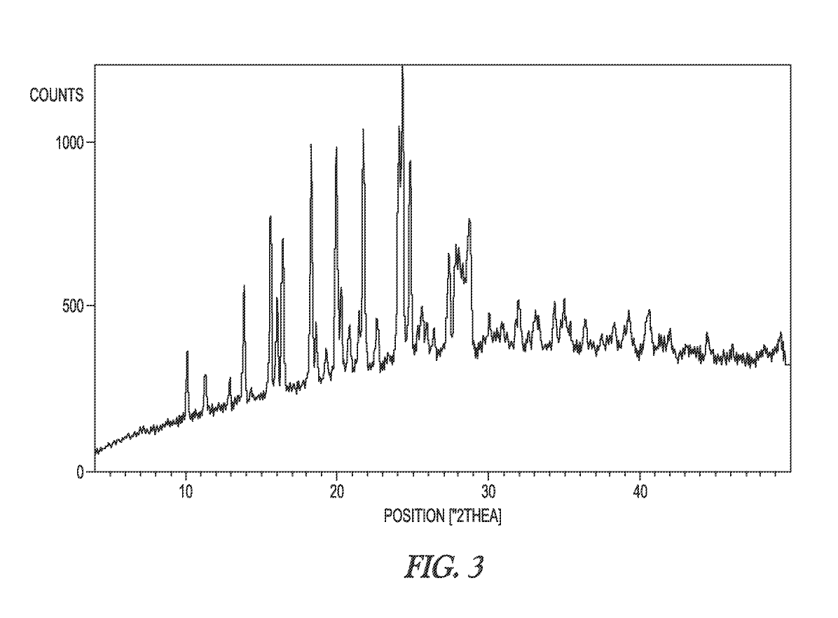

In a preferred embodiment of the present invention, 3-(N-methyl-2(R)-pyrrolidinylmethyl)-5-[2-(phenyl sulfonyl)ethenyl]-1H-indole hydrobromide (II) is obtained in a crystalline form characterized by X-ray diffraction pattern as shown in FIG. 3. The crystalline form of 3-(N-methyl-2(R)-pyrrolidinylmethyl)-5-[2-(phenyl sulfonyl)ethenyl]-1H-indole hydrobromide (II) is characterized by X-ray powder diffraction (XRPD) having characteristic peaks expressed as 2-theta values of about 10.12, 11.34, 13.87, 15.63, 16.09, 16.43, 18.32, 19.95, 20.39, 20.90, 21.82, 22.75, 24.16, 24.37, 24.93, 25.69, 27.47, 27.91 and 28.85 degrees. It is further characterized by peaks expressed as 2-theta of about 32.08, 33.29, 34.45, 36.46, 40.66 and 44.67 degrees.

According to another embodiment of the present invention there is provided 3-(N-methyl-2(R)-pyrrolidinyl methyl)-5-[2-(phenyl sulfonyl)ethenyl]-1H-indole hydrobromide (II), an intermediate used for the preparation of 3-(N-methyl-2(R)-pyrrolidinylmethyl)-5-[2-(phenyl sulfonyl)ethyl]-1H-indole hydrobromide, substantially free of desbromo impurity (V).

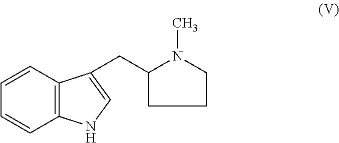

It is found by the inventors of the present invention that when the reaction mixture containing 3-(N-methyl-2(R)-pyrrolidinylmethyl)-5-[2-(phenyl sulfonyl)ethenyl]-1H-indole hydrobromide (II) is subjected to column chromatography, it is obtained as a hygroscopic amorphous solid.

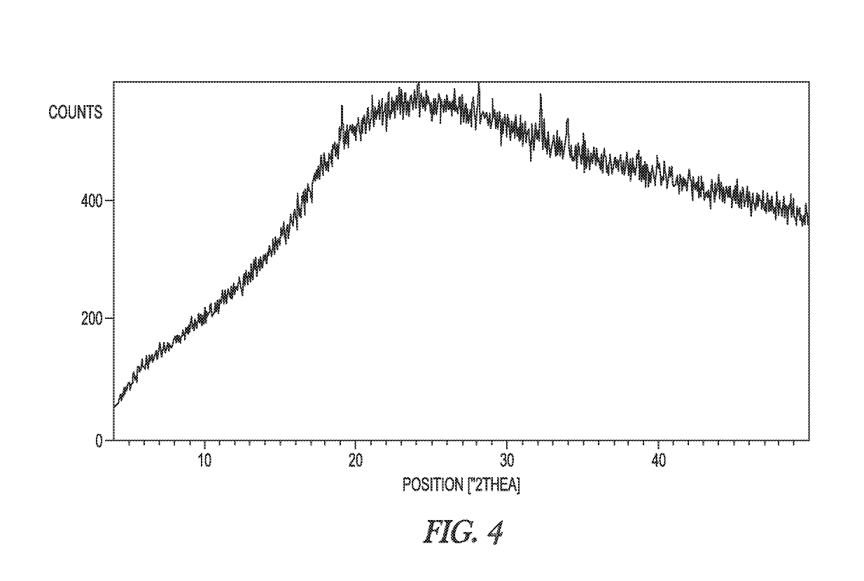

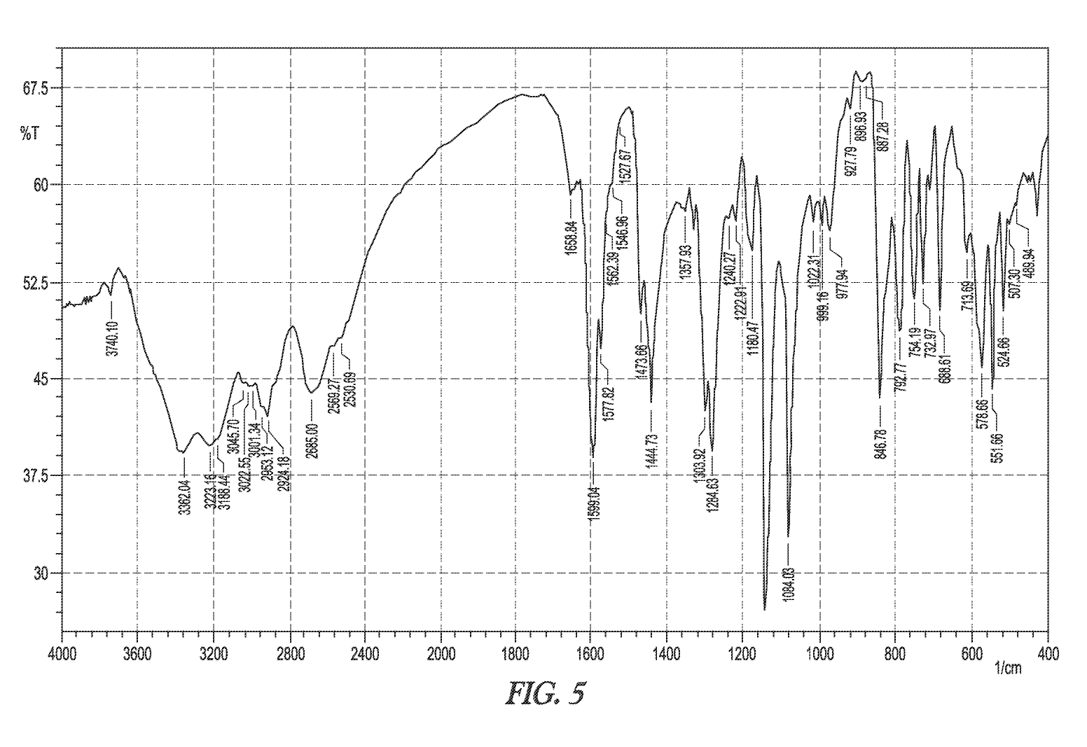

In an alternate preferred embodiment, 3-(N-methyl-2(R)-pyrrolidinylmethyl)-5-[2-(phenyl sulfonyl)ethenyl]-1H-indole hydrobromide (II) is purified using column chromatography to get amorphous 3-(N-methyl-2(R)-pyrrolidinylmethyl)-5-[2-(phenyl sulfonyl)ethenyl]-1H-indole hydrobromide (II) characterized by X-ray powder diffraction (XRPD) as shown in FIG. 4 and/or by an infra-red spectrum as shown in FIG. 5.

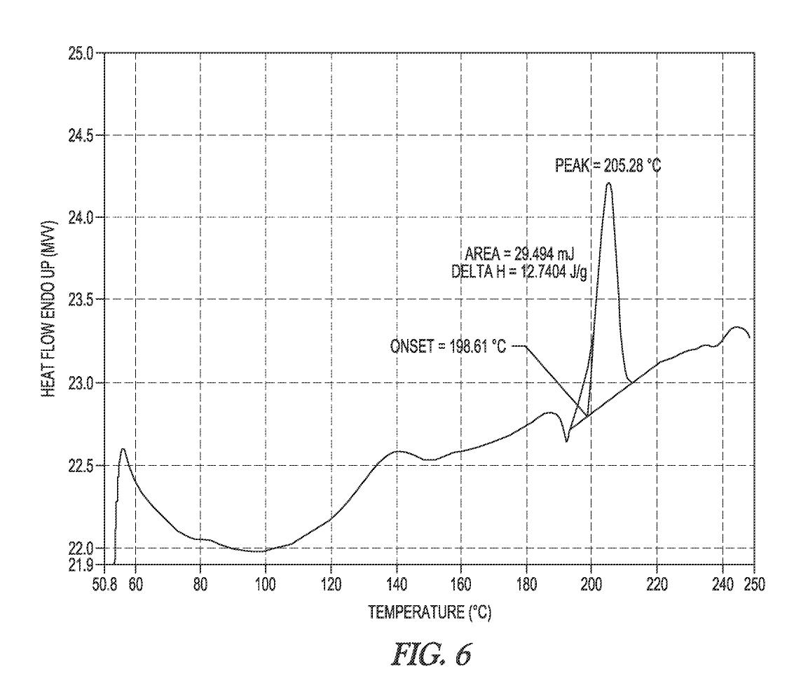

Differential Scanning calorimetry (DSC) of 3-(N-methyl-2(R)-pyrrolidinyl methyl)-5-[2-(phenyl sulfonyl)ethenyl]-1H-indole hydrobromide (II) is as shown in FIG. 6

According to another embodiment, the present invention provides a process for the preparation of 3-(N-methyl-2(R)-pyrrolidinylmethyl)-5-[2-(phenyl sulfonyl)ethyl]-1H-indole hydrobromide (Eletriptan hydrobromide) comprising the steps of,

a) subjecting 3-(N-methyl-2(R)-pyrrolidinylmethyl)-5-[2-(phenyl sulfonyl)ethenyl]-1H-indole hydrobromide (II) to reduction to obtain 3-(N-methyl-2(R)-pyrrolidinyl methyl)-5-[2-(phenyl sulfonyl)ethyl]-1H-indole hydrobromide;
b) optionally, treating 3-(N-methyl-2(R)-pyrrolidinyl methyl)-5-[2-(phenyl sulfonyl)ethyl]-1H-indole hydrobromide obtained in step (a) with HBr in a solvent to obtain 3-(N-methyl-2(R)-pyrrolidinyl methyl)-5-[2-(phenyl sulfonyl)ethyl]-1H-indole hydrobromide with the required HBr content;
c) optionally, purifying 3-(N-methyl-2(R)-pyrrolidinyl methyl)-5-[2-(phenyl sulfonyl)ethyl]-1H-indole hydrobromide to obtain pure 3-(N-methyl-2(R)-pyrrolidinyl methyl)-5-[2-(phenyl sulfonyl)ethyl]-1H-indole hydrobromide compound.

Reduction is carried out in the presence of reducing agent selected from hydrogen gas, hydrides, ammonium formate or formic acid; catalyst selected from palladium, Raney nickel, platinum oxide, rhodium or ruthenium; and solvent selected from C1 to C4 alcohol such as methanol, ethanol, n-propanol, isopropanol or mixture thereof, preferably methanol. Solvent in step (b) is selected from isopropanol, n-propanol, ethanol, methanol, n-butanol or mixture thereof.

In a preferred embodiment, 3-(N-methyl-2(R)-pyrrolidinylmethyl)-5-[2-(phenyl sulfonyl)ethenyl]-1H-indole hydrobromide (II) is catalytically reduced using hydrogen in the presence of a suitable catalyst such as 10% w/w Pd/C at a pressure of about 1 to 10 Kg/cm², preferably 5 Kg/cm² at temperature of 30-50° C., preferably at 40° C. for 6 to 12 hours, preferably for 8 to 10 hours to get a reaction mixture. The obtained reaction mixture is filtered and the filtrate is concentrated to get an oily residue which is treated with HBr in isopropanol (IPA) to obtain 3-(N-methyl-2(R)-pyrrolidinylmethyl)-5-[2-(phenyl sulfonyl)ethyl]-1H-indole hydrobromide with required HBr content. The compound thus obtained is purified. Preferably HBr is used as HBr/isopropanol (IPA) in the concentration of 10 to 30% HBr, preferably 15 to 25% HBr in IPA.

Prior art discloses that the reduction is carried out under a hydrogen atmosphere of 15 psi for 18 hours. In the process of the present invention hydrogenation is carried out at a pressure of about 71 psi (5 Kg/cm²) for 8 to 10 hours. The use of high pressure reduces the reaction time and provides the final product substantially free of impurities.

In a preferred embodiment, 3-(N-methyl-2(R)-pyrrolidinylmethyl)-5-[2-(phenyl sulfonyl)ethyl]-1H-indole hydrobromide obtained after reduction is subjected to solvent treatment followed by treatment with HBr in an organic solvent. The obtained reaction mixture is further heated at 65 to 85° C., preferably at 70° C. followed by cooling the reaction mixture at room temperature, stirring for 30 to 90 minutes, preferably for 60 min. The mixture is further heated, cooled and stirred for 1-3 hours, preferably for 2 hours to precipitate Eletriptan hydrobromide which is filtered and dried to obtain crude 3-(N-methyl-2(R)-pyrrolidinylmethyl)-5-[2-(phenyl sulfonyl)ethyl]-1H-indole hydrobromide.

Solvent treatment involves stripping oily residue with organic solvent to remove the traces of methanol, which provides the final product in high yield and purity. This treatment converts the oily residue into solid which enables easy handling.

Treatment of 3-(N-methyl-2(R)-pyrrolidinylmethyl)-5-[2-(phenyl sulfonyl)ethyl]-1H-indole hydrobromide with HBr enables enrichment of the bromide content of 3-(N-methyl-2(R)-pyrrolidinylmethyl)-5-[2-(phenyl sulfonyl)ethyl]-1H-indole hydrobromide. During the coupling reaction, there is possibility that 3-(N-methyl-2(R)-pyrrolidinylmethyl)-5-[2-(phenyl sulfonyl)ethenyl]-1H-indole hydrobromide is formed with less HBr content. Therefore, isopropanol-HBr is added to enrich the bromide content and to obtain 3-(N-methyl-2(R)-pyrrolidinylmethyl)-5-[2-(phenyl sulfonyl)ethyl]-1H-indole hydrobromide with required HBr content.

In an alternate embodiment of the present invention, 3-(N-methyl-2(R)-pyrrolidinyl methyl)-5-[2-(phenyl sulfonyl)ethenyl]-1H-indole hydrobromide (II) is treated with HBr in a suitable solvent to enrich the bromide content of 3-(N-methyl-2(R)-pyrrolidinyl methyl)-5-[2-(phenyl sulfonyl)ethenyl]-1H-indole hydrobromide (II) which is then reduced to get the desired 3-(N-methyl-2(R)-pyrrolidinyl methyl)-5-[2-(phenyl sulfonyl)ethyl]-1H-indole hydrobromide.

HBr is used as an aqueous, alcoholic or acidic solution. Preferably, HBr in isopropanol or acetic acid is used.

Another embodiment of the present invention provides process for purification of 3-(N-methyl-2(R)-pyrrolidinyl methyl)-5-[2-(phenyl sulfonyl)ethyl]-1H-indole hydrobromide (Eletriptan hydrobromide) which comprises the steps of,

a) dissolving 3-(N-methyl-2(R)-pyrrolidinyl methyl)-5-[2-(phenyl sulfonyl)ethyl]-1H-indole hydrobromide (Eletriptan hydrobromide) in a solvent or mixture of solvents to obtain solution; and
b) optionally treating the obtained solution with neutral alumina and/or activated charcoal to get a mixture; and
c) optionally adding an anti-solvent to the solution of step a) or mixture of step b) to obtain a mixture; and
d) isolating pure 3-(N-methyl-2(R)-pyrrolidinyl methyl)-5-[2-(phenyl sulfonyl)ethyl]-1H-indole hydrobromide (Eletriptan hydrobromide) substantially free of impurities.

Preferably, Eletriptan hydrobromide is dissolved in suitable solvent and the obtained solution is heated at temperature in the range of 50 to 70° C., preferably 55 to 60° C. to get a solution. The obtained solution is treated with neutral alumina and/or activated charcoal maintaining the same temperature to obtain a mixture. The mixture is filtered and the filtrate is concentrated at atmospheric pressure to reduce the volume of solvent to obtain a concentrated mixture. The concentrated mixture is cooled to 25 to 30° C. and stirred for 2 to 10 hours, preferably for 3 to 6 hours to obtain Eletriptan hydrobromide substantially free of impurities.

In an alternate embodiment, crude Eletriptan hydrobromide is dissolved in a suitable solvent to obtain a solution which is refluxed for 1 to 3 hours, preferably for 2 hours at temperature in the range of 50 to 70° C., preferably in the range of 55 to 60° C. to get a clear solution. The obtained clear solution is filtered and the solid obtained is dried to obtain Eletriptan hydrobromide substantially free of impurities.

Suitable solvent used is selected from acetone, methylethyl ketone, methylisobutyl ketone, methanol, ethanol, n-propanol, isopropanol or mixture thereof, preferably acetone or acetone-methanol.

The use of alumina in the purification of the Eletripan hydrobromide helps in the removal of traces of metal impurities such as palladium or platinum metal. The use of alumina also improves the colour of the obtained Eletriptan hydrobromide. Pure Eletriptan hydrobromide obtained has metal content well below 10 ppm.

Prior art discloses the use of 2-propanol for crystallization of Eletriptan hydrobromide. The use of 2-propanol results in the formation of solvate, resulting in high 2-propanol content in Eletriptan hydrobromide, which requires additional steps to reduce the level of 2-propanol. The present invention uses acetone as the solvent for purification, which enables easy removal of the solvent and provides Eletriptan hydrobromide substantially free of impurities.

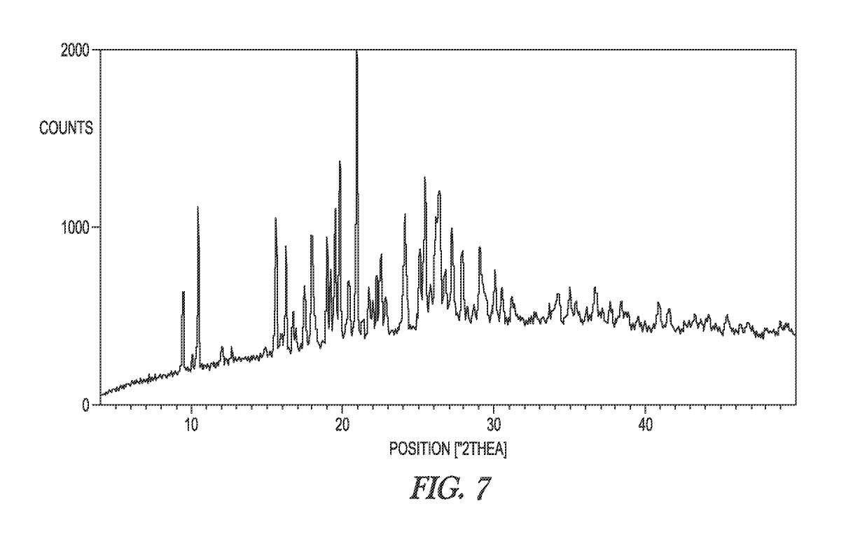

In a preferred embodiment of the present invention, 3-(N-methyl-2(R)-pyrrolidinyl methyl)-5-[2-(phenyl sulfonyl)ethyl]-1H-indole hydrobromide (Eletriptan hydrobromide) obtained is crystalline in nature, preferably alpha form and is characterized by X-ray diffraction pattern as shown in FIG. 7. The alpha form of Eletriptan hydrobromide obtained according to present invention is further characterized by X-ray powder diffraction (XRPD) having peaks at 2-theta values of about 9.48, 10.47, 15.66, 16.29, 17.98, 19.00, 19.54, 19.85 and 20.98 degrees.

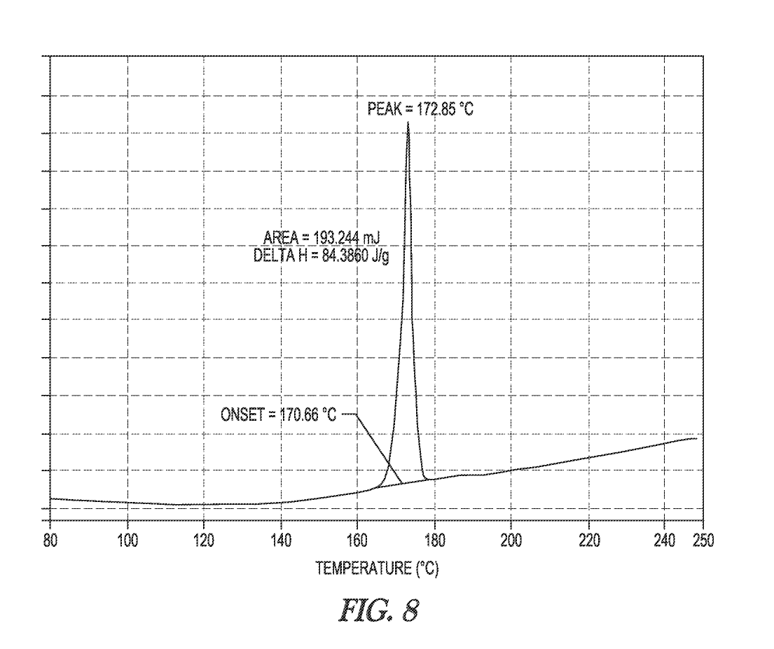

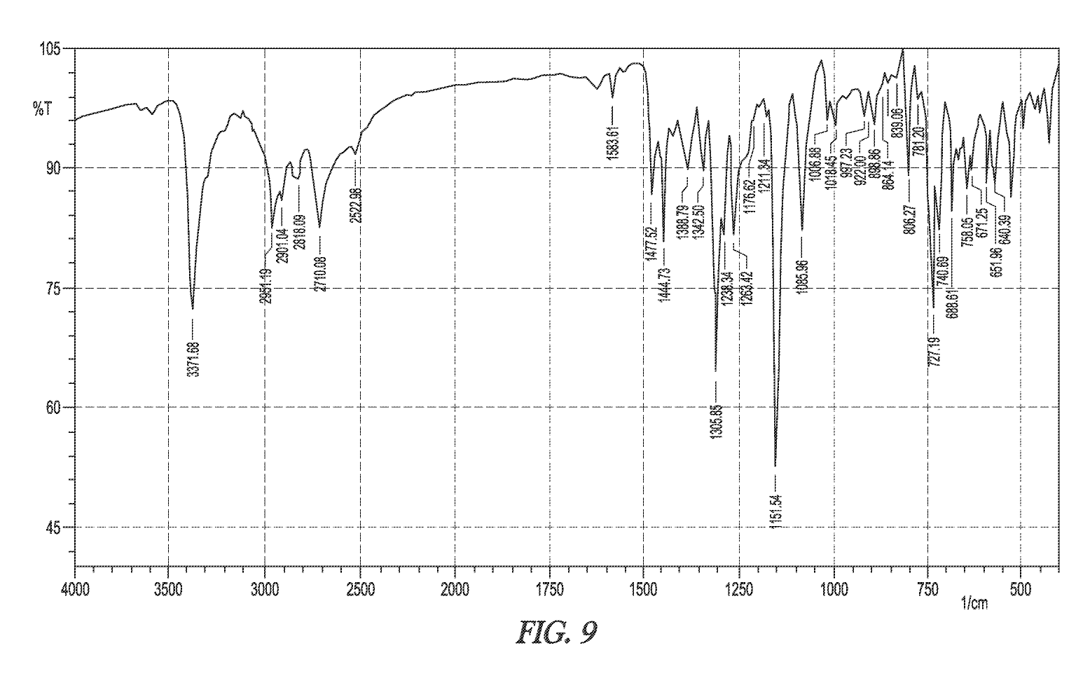

The alpha form of Eletriptan hydrobromide obtained according to present invention is characterized by a Differential Scanning calorimetry (DSC) thermogram having one endothermic peak at 172.85° C., as shown in FIG. 8. The alpha form of Eletriptan hydrobromide obtained according to present invention is characterized by IR spectrum as shown in FIG. 9.

Eletriptan hydrobromide obtained according to the present invention is more than 99.8% pure and has all known impurities below 0.15% and all unknown impurities below 0.1% thus complying the ICH guidelines. Eletriptan hydrobromide obtained according to the present invention has specific optical rotation of +6.0° to +7.0° (c=1, methanol) at 20° C.

Eletriptan hydrobromide obtained according to the present invention is stable, has well defined crystals and good bulk flow properties making it suitable for pharmaceutical formulation.

The process of the present invention has avoided the use of column chromatography. It has been surprisingly found by the inventors of the present invention that by following the process described in U.S. Pat. No. 5,545,644 the overall yield of the final product is only 25%. The process of present invention yields 66% of Eletriptan hydrobromide by avoiding the use of column chromatography making the process commercially viable and cost effective. The present invention is represented in Scheme 2 below,

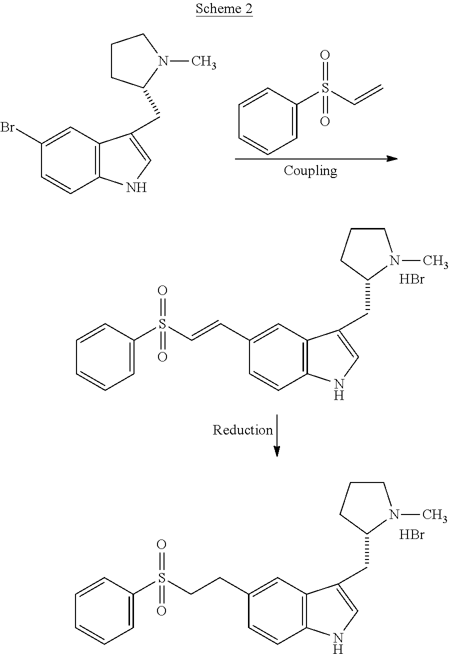

The process for preparation of Eletriptan hydrobromide as described herein can be used to prepare different salts of Eletriptan.

The process of the present invention provides Eletriptan hydrobromide substantially free from impurities, without isolation of Eletriptan base.

In an alternate embodiment of the present invention, Eletriptan base is treated with non-aqueous HBr to obtain Eletriptan hydrobromide. Non-aqueous HBr includes HBr in any organic solvent selected from ketone, alcohol, ester, ether or the like.

Another embodiment of the present invention provides an improved process for preparation of Eletriptan hydrobromide substantially free of genotoxic impurity selected from the group consisting of 2-Chloroethylphenyl sulphide (A), 2-Chloroethylphenyl sulphone (B), Phenyl vinyl sulfone (C), N-Benzyloxycarbonyl-D-proline acid chloride (D) and 3-(N-methyl-2(R)-pyrrolidinyl methyl)-5-[2-(phenyl sulfonyl)ethenyl]-1H-indole (E) well below 19 ppm.

| | | |
|---|---|---|
| 2-Chloroethylphenyl sulphide | 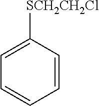 | (A) |
| 2-Chloroethylphenyl sulphone | 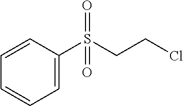 | (B) |
| Phenyl vinyl sulfone | 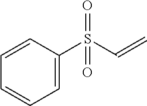 | (C) |
| N-benzyloxycarbonyl-D-proline acid chloride | 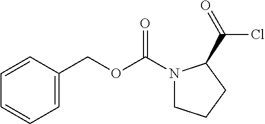 | (D) |
| 3-(N-methyl-2(R)-pyrrolidinyl methyl)-5-[2-(phenyl sulfonyl) ethenyl]-1H-indole | 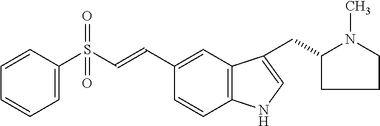 | (E) |

Another embodiment of the present invention provides an improved process for the preparation of Eletriptan hydrobromide substantially free of impurity selected from (3-(1-Methyl pyrrolidin-2(R)-yl methyl)-1H-indole) (F) and 3-[N-Methyl-2(R)-pyrrolidinylmethyl]-1,5-bis-(2-phenylsulfonylethyl)indole (G), which is well below 0.1%.

| | | |
|---|---|---|
| 3-(1-Methyl pyrrolidin-2(R)-yl- methyl)-1H-indole | 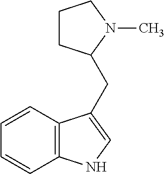 | (F) |
| 3-[N-Methyl-2(R)-pyrrolidinyl methyl]-1,5-bis-(2-phenyl sulfonylethyl)indole | 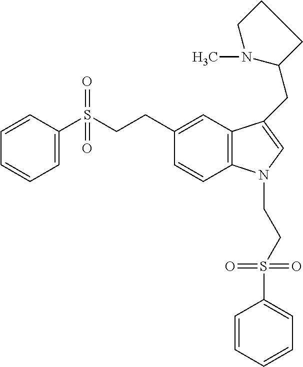 | (G) |

Another embodiment of the present invention provides a process for preparation of 3-[N-Methyl-2(R)-pyrrolidinylmethyl]-1,5-bis-(2-phenylsulfonylethyl)indole, an impurity which can be used as a Reference Marker and/or Reference Standard to determine the purity of a sample of Eletriptan or a pharmaceutical dosage form comprising Eletriptan.

Another embodiment of the present invention provides process for the preparation of 3-[N-Methyl-2(R)-pyrrolidinylmethyl]-1,5-bis-(2-phenylsulfonylethyl)indole, an impurity comprising the steps of,

a) treating Eletriptan base with phenyl vinyl sulfone in the presence of a base and solvent at a temperature of 40-80° C. for about 10-18 hours to obtain a reaction mixture;
b) isolating 3-[N-Methyl-2(R)-pyrrolidinylmethyl]-1,5-bis-(2-phenyl sulfonyl ethyl)indole.

Solvent is selected from N,N-dimethylformamide, N,N-dimethylacetamide, N-methylacetamide, N,N-dimethylpropionamide, preferably N,N-dimethylformamide. Base is selected from sodium hydride, sodium hydroxide, potassium hydroxide, sodium carbonate or potassium carbonate.

In a preferred embodiment, sodium hydride is dissolved in N,N-dimethylformamide and the solution is cooled to 0 to 12° C., preferably 5 to 10° C. Eletriptan base is dissolved in N,N-dimethylformamide and added to the cooled sodium hydride solution at the same temperature to get a reaction mixture. The obtained reaction mixture is stirred at 40° C. to 60° C., preferably at 50° C. for 30 to 90 min, preferably for 60 min. Phenyl vinyl sulfone is dissolved in N,N-dimethylformamide to get a solution which is added to the obtained reaction mixture at 40° C. to 60° C., preferably at 50° C. and stirred at 60° C. to 80° C., preferably at 70° C. for 10 to 17 hours, preferably for 12 to 15 hours. Alcoholic solvent is added to this reaction mixture at 10° C. to 30° C., preferably at 20° C. followed by the addition of water to the reaction mixture. This reaction mixture is then subjected to extraction with an organic solvent, preferably ester. The organic layer is separated, washed with water, dried and concentrated to get an oily product, which is then subjected to column chromatography to obtain 3-[N-Methyl-2(R)-pyrrolidinylmethyl]-1,5-bis-(2-phenyl sulfonyl ethyl)indole.

Alcoholic solvent is selected from methanol, ethanol, isopropanol, n-propanol, butanol or mixture thereof. Ester is selected from methyl acetate, ethyl acetate or butyl acetate, preferably ethyl acetate.

The starting materials, 5-Bromo-3-(1-Methyl pyrrolidin-2(R)-ylmethyl)-1H-indole and phenyl vinyl sulfone are commercial products. 5-Bromo-3-(1-Methyl pyrrolidin-2(R)-ylmethyl)-1H-indole can be prepared by any method known in the art. 5-Bromo-3-(1-methylpyrrolidin-2(R)-ylmethyl)-1H-indole is prepared by a process comprising the steps of reacting D-proline-2-carboxylic acid with benzyl chloroformate in presence of a chlorinating agent to give N-benzyloxycarbonyl-D-proline acid chloride; reacting the obtained acid chloride with 5-bromo indole in presence of a Grignard reagent to yield (R)-3-(N-benzyloxycarbonyl-D-proline-2-ylcarbonyl)-5-bromo-1H-indole, reducing (R)-3-(N-benzyloxycarbonyl-D-proline-2-ylcarbonyl)-5-bromo-1H-indole using suitable reducing agent to provide 5-bromo-3-(1-methylpyrrolidin-2(R)-ylmethyl)-1H-indole.

Chlorinating agent is selected from thionyl chloride, oxalyl chloride, sulfuryl chloride, acetyl chloride or zinc chloride, preferably thionyl chloride. Reducing agent is selected from lithium aluminium hydride, diborane, lithium borohydride and sodium borohydride, preferably lithium aluminium hydride.

Phenyl vinyl sulfone, starting material for the preparation of Eletriptan hydrobromide can be prepared by any process known in the art. It can be prepared by alkylation of thiophenol and 1,2-dihaloethane in presence of base to get β-haloethyl phenyl sulphide, which is then oxidized using oxidizing agent to obtain β-haloethyl phenyl sulfone. The obtained β-haloethyl phenyl sulfone on dehydrohalogenation produces phenyl vinylsulfone.

The base used in the alkylation reaction is selected from sodium hydroxide, potassium hydroxide, potassium tertiary butoxide, sodium carbonate, potassium carbonate, sodium bicarbonate, potassium bicarbonate, liquor ammonia, sodium hydride, calcium or cesium hydroxide. Oxidizing agent used for oxidation is selected from hydrogen peroxide, persulfuric acid, sodium perborate, silver oxide, nitrous oxide, osmium tetroxide or ozone. Halogenated hydrocarbon is selected from chloroform, carbon tetrachloride, methylene chloride (MDC), ethylene dichloride, 1,1,2-ethylenetrichloride or mixture thereof.

Eletriptan hydrobromide obtained according to the present invention has particle size such that about 90% of the particles have particle size less than about 150 microns, preferably less than about 100 microns, more preferably less than about 90 microns. Eletriptan hydrobromide thus obtained can be micronized by conventional techniques to get micronized particles having particle size such that about 90% of the particles have a particle size less than about 20 microns, preferably less than about 10 microns, more preferably less than about 5 microns to enhance the desired properties of the active in the pharmaceutical composition.

According to one embodiment of the present invention, there is provided pharmaceutical compositions comprising Eletriptan or a pharmaceutically acceptable salt thereof and one or more excipients selected from diluents, binders, disintegrants, lubricants, glidants and coating agents. Pharmaceutical compositions of Eletriptan or pharmaceutically acceptable salt thereof may be provided in dosage forms such as tablets, capsules, granules, powders and the like.

Eletriptan hydrobromide prepared by the process as described herein is used for the preparation of pharmaceutical dosage forms such as tablets, capsules, granules, powders and the like. The pharmaceutical compositions of the present invention may be prepared by direct compression method, wet granulation method or slugging-deslugging method.

According to a preferred embodiment of the present invention, the pharmaceutical compositions of Eletriptan hydrobromide are prepared by direct compression method.

According to another embodiment, the process for preparation of pharmaceutical compositions of Eletriptan or a pharmaceutically acceptable salt thereof comprise the steps of,

a) providing Eletriptan or a pharmaceutically acceptable salt thereof;
b) preparing a mixture of Eletriptan or a pharmaceutically acceptable salt thereof, diluents and disintegrants;
c) optionally granulating the mixture of step b) using a binder solution to obtain granules;
d) lubricating the mixture of step b) or granules obtained by step c) using lubricants to obtain lubricated blend;
e) optionally compressing the lubricated blend of step d) into tablets or filling the lubricated blend into capsules;
f) optionally film coating the tablets obtained by step e).

The present invention has the following advantages,
1) Simple, high yielding, commercially viable and cost effective process for preparation of Eletriptan hydrobromide.
2) Does not involve isolation of Eletriptan base.
3) Avoids the use of column chromatography for purification.
4) Present invention provides the process for preparation of Eletriptan hydrobromide substantially free of impurities.
5) Reduction of cycle time.
6) Easy scale-up and consistent process for preparation of Eletriptan hydrobromide.
7) Use of alumina helps in eliminating the palladium content and reducing the colour of the final product.

Unless otherwise indicated, the following definitions are set forth to illustrate and define the meaning and scope of the various terms used to describe the invention herein.

The term “substantially free” means a compound having less than about 1%, preferably less than about 0.5%, more preferably less than about 0.3%, most preferably less than about 0.15% of undesired impurities or other polymorphic forms.

The term “reflux temperature” means the temperature at which the solvent or solvent system refluxes or boils at atmospheric pressure.

The term “room temperature” means a temperature from about 10° C. to 45° C., preferably 25° C. to 30° C.

Identification of solid obtained by the present invention can be made by methods known in the art such as X-Ray powder diffraction (XRPD), Fourier Transform Infrared (FT-IR) spectra, and differential scanning calorimetry (DSC).

The XRPD, FT-IR and DSC and methods used for the identification and characterization of Eletriptan, salt thereof or intermediate thereof are described below:

a) FT-IR Spectral Analysis: IR spectra was recorded on SHIMADZU in the energy range of 4000 to 400 cm⁻¹
b) X-ray powder diffraction: XRPD Spectrum was recorded on PANalytical X′pert Pro diffractometer equipped with accelerator detector using Copper K□ (□□1.5406 Å) radiation with scanning range between 4-50 2□□ at scanning speed of 2°/min.
c) DSC: DSC analysis was recorded on Perkin Elmer at a heating rate of 20° C. per minute at a temperature range from 50° C. to 250° C.

The following examples are for illustrative purposes only and are not intended, nor should they be interpreted, to limit the scope of the invention.

### EXAMPLES

### Example 1

### Preparation of 3-(N-methyl-2(R)-pyrrolidinyl methyl)-5-[2-(phenyl sulfonyl)ethenyl]-1H-indole hydrobromide (II)

Acetonitrile(2 L), 5-Bromo-3-(1-Methyl pyrrolidin-2(R)-yl methyl)-1H-indole (200 g, 0.68 mol), phenyl vinyl sulfone (152 g, 0.90 mol), tri-o-tolyl phosphine (30 g, 0.098 mol), palladium acetate (5 g, 0.022 mol) and triethyl amine (200 ml, 1.58 mol) were successively charged to a reactor to get reaction mixture. The obtained reaction mixture was stirred at 75 to 80° C. for 8 to 10 hours. After completion of reaction, the reaction mixture was filtered through a hyflo bed. The obtained filtrate was concentrated under vacuum to obtain a residue which was stripped with acetonitrile (2×400 ml) to obtain a solid. 1 L of acetonitrile was added to the solid and the mixture was stirred at 25 to 30° C. for 2 hours. The solid thus obtained was filtered and dried to get compound (II).

Yield: 244 g, 78%; Purity 95%.

### Example 2

### Purification of 3-(N-methyl-2(R)-pyrrolidinyl methyl)-5-[2-(phenyl sulfonyl)ethenyl]-1H-indole hydrobromide

240 g of 3-(N-methyl-2(R)-pyrrolidinyl methyl)-5-[2-(phenyl sulfonyl)ethenyl]-1H-indole hydrobromide was dissolved in 2.4 L of toluene and stirred for 2 hours at 70° C. to get a solution. 480 ml of ethanol was slowly added at 70° C. to obtain a mixture. The mixture was stirred for 2 hours at 70 to 75° C. The mixture was then cooled gradually to 25 to 30° C. and stirred for 12 hours to obtain a solid. The solid thus obtained was filtered, washed with toluene and dried to yield (3-(N-methyl-2(R)-pyrrolidinyl methyl)-5-[2-(phenyl sulfonyl)ethenyl]-1H-indole hydrobromide.

Yield: 207 g, 85%; Purity 97%

### Example 3

### Purification of 3-(N-methyl-2(R)-pyrrolidinyl methyl)-5-[2-(phenyl sulfonyl)ethenyl]-1H-indole hydrobromide

200 g of 3-(N-methyl-2(R)-pyrrolidinyl methyl)-5-[2-(phenyl sulfonyl)ethenyl]-1H-indole hydrobromide was dissolved in 2 L of toluene and stirred for 2 hours at 70° C. to get a solution. 400 ml of ethanol was slowly added at 70° C. to obtain a mixture. The mixture was stirred for 2 hours at 70 to 75° C. The mixture was then cooled gradually at 25 to 30° C. and stirred for 12 hours to obtain a solid. The solid thus obtained was filtered, washed with toluene and dried to yield 3-(N-methyl-2(R)-pyrrolidinyl methyl)-5-[2-(phenyl sulfonyl)ethenyl]-1H-indole hydrobromide.

Yield: 191 g, 96%; Purity 98%

### Example 4

### Preparation of 3-(N-methyl-2(R)-pyrrolidinyl methyl)-5-[2-(phenyl sulfonyl)ethyl]-1H-indole hydrobromide (Eletriptan hydrobromide) (I) crude

190 gm of 3-(N-methyl-2(R)-pyrrolidinyl methyl)-5-[2-(phenyl sulfonyl)ethenyl]-1H-indole hydrobromide was dissolved in 1.9 L of methanol and charged in a hydrogenator. 190 ml of methanol was added to the mixture and 10% Palladium on carbon (76 gm) were carefully added to the hydrogenator. Hydrogenation was carried out at a pressure of 5 Kg/cm² at 40° C. for 8 to 10 hours. After completion of reaction, the reaction mixture was filtered through hyflo bed and the filtrate was concentrated to get an oily residue. The residue thus obtained was stripped with isopropanol (2×400 ml). 950 ml of isopropanol (IPA) was added to the obtained residue followed by addition of 138 ml of HBr/isopropanol. The reaction mixture was heated to 70° C., cooled to 25° C. to 30° C. and stirred for 1 hour followed by further heating the obtained mixture at 75-80° C. for 2 hours. The mixture was cooled to 25° C. to 30° C. and stirred for 2 hours to get a solid. The solid obtained was filtered and dried to get Eletriptan hydrobromide crude.

Yield: 168 g, 88.33%; Purity 98 to 99%

### Example 5

### Purification of 3-(N-methyl-2(R)-pyrrolidinyl methyl)-5-[2-(phenyl sulfonyl)ethyl]-1H-indole hydrobromide (Eletriptan hydrobromide) (I)

165 gm of crude 3-(N-methyl-2(R)-pyrrolidinyl methyl)-5-[2-(phenyl sulfonyl)ethyl]-1H-indole hydrobromide (I) in acetone (24.75 L) was refluxed at 55° to 60° C. till a clear solution was obtained. Neutral alumina (165 gm) and activated charcoal (16.5 gm) were added to the obtained clear solution and the mixture was refluxed for 1 hour. The hot solution was filtered through hyflo bed and obtained filtrate was concentrated at atmospheric pressure. During concentration, about 23.10 L of solvent was distilled out. The mixture was cooled to 25 to 3° C. and stirred for 8 to 10 hours to get the solid. The solid obtained was filtered and dried to get pure compound (I).

Yield: 124 g, 75%; Purity 99.8%

### Example 6

### Purification of 3-(N-methyl-2(R)-pyrrolidinyl methyl)-5-[2-(phenyl sulfonyl)ethyl]-1H-indole hydrobromide (Eletriptan hydrobromide) (I)

165 gm of crude 3-(N-methyl-2(R)-pyrrolidinyl methyl)-5-[2-(phenyl sulfonyl)ethyl]-1H-indole hydrobromide (I) in acetone (1.65 L) was refluxed for 2 hours at 55° C. to 60° C. The mixture was filtered at the same temperature to get solid. The solid obtained was filtered and dried to get pure compound (I).

Yield: 140 g, 85%; Purity 99.7%

### Example 7

### Preparation of 3-[N-Methyl-2(R)-pyrrolidinylmethyl]-1,5-bis-(2-phenyl sulfonyl ethyl)indole (Impurity at RRT 1.9):

5 gm of sodium hydride was dissolved in 50 ml of dimethyl formamide and cooled to 5 to 10° C. to get solution. 30 gm of Eletriptan base was dissolved in dimethyl formamide and was added to the cooled solution at 5 to 10° C. to get reaction mixture. The reaction mixture was stirred at 50° C. for 1 hour. 15.8 gm of phenyl vinyl sulphone was dissolved in 30 ml of dimethyl formamide and added to the reaction mixture at 50° C. The reaction mixture was stirred at 70° C. for 12 to 15 hours. 25 ml of methanol at 20° C. was added to obtained reaction mixture followed by addition of 300 ml of water. The reaction mass was extracted with ethyl acetate (3×200 ml). The separated organic layer was washed with water (3×100 ml). The obtained solution was dried over sodium sulphate. The solvent was distilled off to get an oil which was chromatographed on silica gel. Elution with chloroform:methanol (8:2) gave the titled compound as solid.

Yield: 22 gm; Purity 95%

### Example 8

### Preparation of 3-[(R)—N-Benzyloxycarbonylpyrrolidin-2-ylcarbonyl)]-5-bromo-1H-indole

A solution of N-benzyloxy-carbonyl-D-Proline (5 g) in anhydrous dichloromethane (10 ml) was stirred at 25-30° C. for 1.5 hours followed by addition of N,N-dimethylformamide (2 drops) and oxalyl chloride (1 ml) to obtain a reaction mass. The solvent was evaporated under reduced pressure to give the N-benzyloxycarbonyl-D-proline acid chloride. A solution of 3 M ethyl magnesium bromide in ether (2.8 ml) was added dropwise over 5 minutes to a stirred solution of 5-bromoindole (1.5 g) in dry ether (36 ml) The mixture was stirred at 25-30° C. for 10 minutes and further heated under reflux for 2 hours. The reaction mixture was cooled to −30° C. and a solution of N-benzyloxycarbonyl-D-proline acid chloride in dry ether (8 ml) was added dropwise to it. The mixture was stirred for 1 hour followed by addition of ether (25 ml) and saturated aqueous sodium bicarbonate solution (13 ml). The temperature of the reaction mixture was allowed to rise to 25-30° C. with continued stirring for 10 minutes. The mixture was filtered and the obtained filtrate was washed with brine and then dried over magnesium sulphate. The solvent was evaporated to get an oil which was chromatographed on silica gel (elution with ethyl acetate) to get the title compound as a foam.

Yield: 1.2 g, 38%.

### Example 9

### Preparation of 3-(N-methyl-2(R)-pyrrolidinylmethyl)-5-bromo-1H-indole

A solution of 3-[(R)—N-Benzyloxycarbonylpyrrolidin-2-ylcarbonyl)]-5-bromo-1H-indole (1.0 g) in dry tetrahydrofuran (19.2 ml) was added dropwise to a stirred suspension of lithium aluminium hydride (0.26 g) in dry tetrahydrofuran (14.4 ml) at 25-30° C. under an atmosphere of dry nitrogen. The mixture was heated under reflux with stirring for 18 hours and then cooled to 25-30° C. Additional lithium aluminium hydride (48.1 mg) was added and refluxing was continued for an additional 3 hours. The mixture was further cooled and lithium aluminium hydride (38.5 mg) was added. Refluxing was continued for further 18 hours. The mixture was cooled and then successively water (0.42 ml), 20% aqueous sodium hydroxide (0.42 ml) and water (1.28 ml) were added to the mixture. The mixture was diluted with ethyl acetate and filtered through a celite bed. The filtrate was washed with water, followed by brine and then dried over sodium sulphate. Evaporation of the solvent under reduced pressure gave an oil which was chromatographed on silica gel. Elution with dichloromethane:ethanol:concentrated aqueous ammonia (90:10:0.5) gave the title compound as a solid.

Yield: 0.4 g, 56%.

### Example 10

### Preparation of 3-(N-methyl-2(R)-pyrrolidinyl methyl)-5-[2-(phenyl sulfonyl)ethenyl]-1H-indole hydrobromide (II)

A mixture of palladium acetate (1.25 g, 0.0055 mol) and tri-o-tolyl phosphine (3.75 g, 0.0123 mol) in dimethylformamide (100 ml) was stirred under nitrogen atmosphere. 3-(N-methyl-2(R)-pyrrolidinylmethyl)-5-bromo-1H-indole (50 g, 0.17 mol), phenyl vinyl sulfone (28 g, 0.17 mol), triethyl amine (35 ml, 0.27 mol) and dimethyl formamide (150 ml) were added successively to the obtained reaction mixture and the mixture was heated at 90 to 95° C. after the completion of the reaction, the reaction mixture was cooled to 10 to 15° C. and filtered through a hyflo bed. The obtained filtrate was concentrated under vacuum to get a thick oil which was chromatographed using silica gel [Eluent: MDC:Methanol (95:5)] to get amorphous compound (II).

Yield: 35 g, 47%; Purity 92%

### Example 11

### Preparation of 3-(N-methyl-2(R)-pyrrolidinyl methyl)-5-[2-(phenyl sulfonyl)ethyl]-1H-indole hydrobromide (Eletriptan hydrobromide) (I) crude

A mixture of 3-(N-methyl-2(R)-pyrrolidinyl methyl)-5-[2-(phenyl sulfonyl)ethenyl]-1H-indole hydrobromide (II) (35 g, 0.075 mol) in methanol (525 ml) and 10% Palladium on carbon (14 g) was hydrogenated in an autoclave at 70 psi at 40° C. to 45° C. for 8 h. After completion of reaction, the reaction mixture was filtered through a hyflow bed and methanol was evaporated under vacuum to get an oil. The oily residue was stirred in 100 ml isopropanol at 25 to 30° C. for half an hour. The solution thus obtained was refluxed for two hours followed by gradual cooling to room temperature to get a solid. The solid obtained was filtered and dried to get Eletriptan hydrobromide crude.

Yield: 28 g, 80%; Purity 96%

### Example 12

### Purification of 3-(N-methyl-2(R)-pyrrolidinyl methyl)-5-[2-(phenyl sulfonyl)ethyl]-1H-indole hydrobromide (Eletriptan hydrobromide) (I)

### Method I:

A mixture of crude 3-(N-methyl-2(R)-pyrrolidinyl methyl)-5-[2-(phenyl sulfonyl)ethyl]-1H-indole hydrobromide (I) (28 g, 0.06 mol) in acetone (588 ml) and IPA (252 ml) was refluxed at 65 to 70° C. till a clear solution was formed. The hot solution was filtered through a hyflow bed and the obtained filtrate was concentrated at atmospheric pressure. During concentration, about 580 ml of solvent was distilled out and the remaining residue was gradually cooled to 45° C. 14 ml of IPA-HBr was added to the cold residue and the mixture was further cooled to 25 to 30° C. followed by stirring for 4 h. The solid obtained was filtered and dried.

Yield: 22.4 g, 80%

### Method II:

A mixture of 3-(N-methyl-2(R)-pyrrolidinyl methyl)-5-[2-(phenyl sulfonyl)ethyl]-1H-indole hydrobromide (I) (22.4 g) in acetone (540 ml) and IPA (232 ml) was refluxed at 65 to 70° C. till a clear solution was obta fined. Neutral alumina (22.4 g) and activated charcoal (2.24 g) were added to the obtained clear solution and the mixture was refluxed for one hour. The hot solution was filtered through a hyflo bed and the obtained filtrate was concentrated at atmospheric pressure. During concentration, about 500 ml of solvent was distilled out. The mixture was cooled to 25 to 30° C. and stirred for 3 hours to get the solid. The solid obtained was filtered and dried.

Yield: 17.9 g, 80%.

### Example 13

### Preparation of 3-(N-methyl-2(R)-pyrrolidinyl methyl)-5-[2-(phenyl sulfonyl)ethyl]-1H-indole hydrobromide (Eletriptan hydrobromide alpha form)

A mixture of (1 g, 52 mmol) of 3-(N-methyl-2(R)-pyrrolidinyl methyl)-5-[2-(phenyl sulfonyl)ethyl]-1H-indole hydrobromide, 5 ml of acetone and 2 ml of methanol was cooled to 0-5° C. Hydrobromic acid in acetic acid (1.3 ml, 52 mmol) was added to the obtained solution and stirred for 1 hour at 0-5° C. The solution was further stirred at 30° C. for 3 hour to get the alpha form of Eletriptan hydrobromide.

Yield: 0.6 g, 50%.

### Example 14

### Preparation of 3-(N-methyl-2(R)-pyrrolidinyl methyl)-5-[2-(phenyl sulfonyl)ethyl]-1H-indole hydrobromide (Eletriptan hydrobromide alpha form)

A mixture of 3-(N-methyl-2(R)-pyrrolidinyl methyl)-5-[2-(phenyl sulfonyl)ethyl]-1H-indole hydrobromide (2.0 g, 104 mmol), acetone (60 ml) and methanol (6 ml) was stirred at 65° C. for 1 hour. 30 ml of solvent was distilled out from the mixture. The mixture was cooled to 3° C. and stirred for 1 hour. The obtained solid was filtered, washed with chilled acetone (10 ml) and dried.

Yield: 1.30 g, 65%

### Example 15

### Preparation of 3-(N-methyl-2(R)-pyrrolidinyl methyl)-5-[2-(phenyl sulfonyl)ethyl]-1H-indole hydrobromide (Eletriptan hydrobromide alpha form)

A mixture of 3-(N-methyl-2(R)-pyrrolidinyl methyl)-5-[2-(phenyl sulfonyl)ethyl]-1H-indole hydrobromide (5 g, 10 mmol), methanol (60 ml), acetic acid (30 ml) and 10% palladium on carbon (5 g) was shaken under a hydrogen atmosphere (70 psi) at 40° C. for 6 hours. The reaction mixture was filtered over celite filter bed and the obtained residue was washed with methanol. The combined filtrate and washings were evaporated under reduced pressure to get oily compound which was stripped out with IPA to get the solid. The obtained solid was stirred with IPA at 70° C. for 1 hour and cooled to 30° C. to get solid which was filtered and dried.

Yield: 3.2 g, 64%

### Example 16

### Preparation of 3-(N-methyl-2(R)-pyrrolidinyl methyl)-5-[2-(phenyl sulfonyl)ethyl]-1H-indole hydrobromide (Eletriptan hydrobromide alpha form)

3-(N-methyl-2(R)-pyrrolidinyl methyl)-5-[2-(phenyl sulfonyl)ethyl]-1H-indole hydrobromide (10.0 g, 520 mmol) was dissolved in 200 ml of acetone. 400 ml IPA was added to the above solution and stirred for 1 hour at 20-25° C. The obtained solution was further stirred at 30° C. for 3 hours to get the titled product which was filtered and dried.

Yield: 4.2 g, 42%.

### Example 17

### Preparation of 3-(N-methyl-2(R)-pyrrolidinyl methyl)-5-[2-(phenyl sulfonyl)ethyl]-1H-indole hydrobromide (Eletriptan hydrobromide) (I)

35 gm of 3-(N-methyl-2(R)-pyrrolidinyl methyl)-5-[2-(phenyl sulfonyl)ethenyl]-1H-indole hydrobromide was dissolved in 700 ml of methanol and charged in a hydrogenator. 190 ml of methanol was added to the mixture and 10% Palladium on carbon (7 gm) were carefully added to the hydrogenator. Hydrogenation was carried out at a pressure of 5 Kg/cm² at 40° C. for 8 to 10 hours. After completion of reaction, the reaction mixture was filtered through hyflo bed and the filtrate was concentrated to get an oily residue. The residue thus obtained was stripped with methanol (2×300 ml) followed by addition of 250 ml of acetone at 25 to 30° C. The mixture was stirred at the same temperature for 3 to 4 hours to obtain Eletriptan hydrobromide.

### Example 18

### Eletriptan Hydrobromide Tablets

Eletriptan hydrobromide (24.7 g), microcrystalline cellulose (Avicel PH 112®) (147 g) lactose (supertab 21®) (23 g) and croscarmellose sodium (Ac-di-sol®) (4 g) were sifted using 40 mesh s.s.sieve and mixed in a conta blender for sufficient time. Magnesium stearate (pre-sifted using 60 mesh s.s. sieve) (2 g) was added to the dry mix and lubricated for sufficient time. The lubricated blend was compressed into round biconvex core tablets of Eletriptan hydrobromide 20 mg. The core tablets were film coated using ready mix coating material containing hypromellose, lactose monohydrate, triacetin, titanium dioxide and appropriate colours.

The complete disclosure of all patents, patent documents and publications cited herein are incorporated herein by reference as if individually incorporated. The foregoing detailed description and examples have been given for clarity of understanding only. No unnecessary limitations are to be understood therefrom. The invention is not limited to the exact details shown and described for variations obvious to one skilled in the art will be included within the invention defined by the claims.

What is claimed is:

1. A process for preparation of Eletripan hydrobromide comprising,
a) coupling 3-(N-methyl-2(R)-pyrrolidinylmethyl)-5-bromo-1H-indole with phenyl vinyl sulfone to obtain a mixture containing 3-(N-methyl-2(R)-pyrrolidinylmethyl)-5-[2-(phenylsulfonyl)ethenyl]-1H-indole hydrobromide;
b) isolating 3-(N-methyl-2(R)-pyrrolidinylmethyl)-5-[2-(phenylsulfonyl)ethenyl]-1H-indole hydrobromide as a solid;
c) converting said solid 3-(N-methyl-2(R)-pyrrolidinylmethyl)-5-[2-(phenylsulfonyl)ethenyl]-1H-indole hydrobromide to 3-(N-methyl-2(R)-pyrrolidinylmethyl)-5-[2-(phenylsulfonyl)ethyl]-1H-indole hydrobromide (Eletriptan hydrobromide).

2. The process as claimed in claim 1, wherein said coupling in step a) is carried out at a temperature of about 70 to 85° C. for 6 to 14 hours in the presence of an organopalladium catalyst selected from palladium acetate, palladium chloride or tetrakis(phenylphosphine)palladium(0); a triarylphosphine selected from tri-o-tolylphosphine, triphenylphosphine or BINAP; base selected from triethylamine, trimethyl amine, potassium carbonate, sodium carbonate, sodium acetate or potassium acetate; and solvent selected from acetonitrile, N,N-dimethylformamide or tetrahydrofuran.

3. The process as claimed in claim 1 or 2, wherein said coupling in step a) is carried out at a temperature of 80 to 82° C. for 8 to 12 hours in presence of palladium acetate, tri-o-tolylphosphine, triethylamine and acetonitrile.

4. The process as claimed in claim 1, wherein said isolation comprises the steps of,
a) filtering said mixture containing 3-(N-methyl-2(R)-pyrrolidinylmethyl)-5-[2-(phenylsulfonyl)ethenyl]-1H-indole hydrobromide followed by concentrating the filtrate under vacuum to obtain a residue;
b) treating said residue with a solvent to obtain 3-(N-methyl-2(R)-pyrrolidinylmethyl)-5-[2-(phenylsulfonyl)ethenyl]-1H-indole hydrobromide as solid;
c) optionally, purifying said obtained 3-(N-methyl-2(R)-pyrrolidinylmethyl)-5-[2-(phenylsulfonyl)ethenyl]-1H-indole hydrobromide;
d) converting said 3-(N-methyl-2(R)-pyrrolidinylmethyl)-5-[2-(phenyl sulfonyl)ethenyl]-1H-indole hydrobromide from step (b) or step (c) to 3-(N-methyl-2(R)-pyrrolidinylmethyl)-5-[2-(phenylsulfonyl)ethyl]-1H-indole hydrobromide (Eletriptan hydrobromide).

5. The process as claimed in claim 4, wherein solvent used in step b) is a polar aprotic solvent selected from acetonitrile, N,N-dimethylformamide, dimethylsulfoxide, dichloromethane, ethyl acetate, acetone or tetrahydrofuran.

6. The process as claimed in claim 4, wherein said purification in step c) comprises the steps of,
a) dissolving 3-(N-methyl-2(R)-pyrrolidinylmethyl)-5-[2-(phenyl sulfonyl)ethenyl]-1H-indole hydrobromide in first solvent to obtain a solution;
b) optionally adding second solvent to said solution to obtain a mixture; and
c) isolating pure 3-(N-methyl-2(R)-pyrrolidinylmethyl)-5-[2-(phenyl sulfonyl)ethenyl]-1H-indole hydrobromide from said solution or said mixture.

7. The process as claimed in claim 6, wherein said first solvent is selected from toluene, xylene, benzene, hexane, heptane, halogenated derivative thereof, methanol, ethanol, isopropanol, n-propanol, butanol or mixture thereof; and said second solvent is selected from methanol, ethanol, isopropanol, n-propanol, butanol, dichloromethane, N,N-dimethylformamide, ethyl acetate, acetonitrile, tetrahydrofuran, dimethylsulfoxide or mixture thereof.

8. The process as claimed in claim 1, wherein said isolated 3-(N-methyl-2(R)-pyrrolidinylmethyl)-5-[2-(phenyl sulfonyl)ethenyl]-1H-indole hydrobromide is crystalline and is characterized by X-ray diffraction pattern having peaks at 2-theta values of about 11.47, 12.63, 14.41, 15.09, 18.42, 19.77, 22.22, 23.18, 23.90, 25.23, 26.94, 28.33, 29.90 and 30.85 degrees; or by X-ray diffraction pattern having peaks at 2-theta values of about 10.12, 11.34, 13.87, 15.63, 16.09, 16.43, 18.32, 19.95, 20.39, 20.90, 21.82, 22.75, 24.16, 24.37, 24.93, 25.69, 27.47, 27.91 and 28.85 degrees.

9. 3-(N-methyl-2(R)-pyrrolidinylmethyl)-5-[2-(phenylsulfonyl)ethenyl]-1H-indole hydrobromide characterized by X-ray diffraction pattern having peaks at 2-theta values of about 11.47, 12.63, 14.41, 15.09, 18.42, 19.77, 22.22, 23.18, 23.90, 25.23, 26.94, 28.33, 29.90 and 30.85 degrees; or by X-ray diffraction pattern having peaks at 2-theta values of about 10.12, 11.34, 13.87, 15.63, 16.09, 16.43, 18.32, 19.95, 20.39, 20.90, 21.82, 22.75, 24.16, 24.37, 24.93, 25.69, 27.47, 27.91 and 28.85 degrees.

10. The process as claimed in claim 1 or 4, wherein said conversion comprises the steps of,
a) subjecting 3-(N-methyl-2(R)-pyrrolidinylmethyl)-5-[2-(phenylsulfonyl)ethenyl]-1H-indole hydrobromide to reduction to obtain 3-(N-methyl-2(R)-pyrrolidinylmethyl)-5-[2-(phenylsulfonyl)ethyl]-1H-indole hydrobromide;
b) treating 3-(N-methyl-2(R)-pyrrolidinylmethyl)-5-[2-(phenylsulfonyl)ethyl]-1H-indole hydrobromide obtained in step (a) with HBr in a solvent selected from isopropanol, n-propanol, ethanol, methanol, n-butanol or mixture thereof to obtain 3-(N-methyl-2(R)-pyrrolidinyl methyl)-5-[2-(phenylsulfonyl)ethyl]-1H-indole hydrobromide with the required HBr content;
c) optionally purifying said 3-(N-methyl-2(R)-pyrrolidinyl methyl)-5-[2-(phenylsulfonyl)ethyl]-1H-indole hydrobromide to obtain pure 3-(N-methyl-2(R)-pyrrolidinyl methyl)-5-[2-(phenylsulfonyl)ethyl]-1H-indole hydrobromide.

11. The process as claimed in claim 10, wherein said reduction is carried out at a temperature of about 30° C. to 50° C. and pressure of 1 to 10 Kg/cm² for 6 to 12 hours in the presence of hydrogen gas; catalyst selected from palladium, platinum oxide, rhodium, ruthenium or Raney nickel; and solvent selected from methanol, ethanol, n-propanol or isopropanol.

12. A process for preparation of Eletriptan hydrobromide, wherein the process comprises treating 3-(N-methyl-2(R)-pyrrolidinyl methyl)-5-[2-(phenyl sulfonyl)ethyl]-1H-indole hydrobromide having inadequate HBr content with HBr to obtain Eletriptan hydrobromide with required hydrobromide content.

13. The process as claimed in claim 10, wherein said purification in step c) comprises the steps of,
a) dissolving said 3-(N-methyl-2(R)-pyrrolidinyl methyl)-5-[2-(phenyl sulfonyl)ethyl]-1H-indole hydrobromide in a solvent or mixture of solvents to obtain a solution;
b) optionally treating said obtained solution with neutral alumina and/or activated charcoal to get a mixture; and
c) isolating pure 3-(N-methyl-2(R)-pyrrolidinylmethyl)-5-[2-(phenyl sulfonyl)ethyl]-1H-indole hydrobromide (Eletriptan hydrobromide) substantially free of impurities.

14. The process as claimed in claim 13, wherein said solvent is selected from acetone, methylethylketone, methylisobutylketone, methanol, ethanol, n-propanol, isopropanol, butanol or mixture thereof.

15. 3-[N-Methyl-2(R)-pyrrolidinylmethyl]-1,5-bis-(2-phenylsulfonylethyl)indole prepared by a process comprising treating 3-(N-methyl-2(R)-pyrrolidinylmethyl)-5-[2-(phenyl sulfonyl)ethyl]-1H-indole (Eletriptan base) with phenylvinyl sulfone in presence of a base.

16. 3-[N-Methyl-2(R)-pyrrolidinylmethyl]-1,5-bis-(2-phenylsulfonylethyl)indole as claimed in claim 15 for use as a reference marker and/or reference standard in testing the purity of a sample of Eletriptan or a pharmaceutical dosage form comprising Eletriptan.
